(19) United States
(12) Patent Application Publication
(10) Pub. No.: US 2005/0144667 A1
(43) Pub. Date: Jun. 30, 2005

(54) Plant polypeptides and polynucleotides encoding same

(76) Inventors: Duncan Stanley, Auckland (NZ); Elspeth Macrae, Auckland (NZ)

Correspondence Address: GREENLEE WINNER AND SULLIVAN P C, 4875 PEARL EAST CIRCLE, SUITE 200, BOULDER, CO, 80301, US

(21) Appl. No.: 10/490,928

(22) PCT Filed: Sep. 30, 2002
(86) PCT No.: PCT/NZ02/00200

(30) Foreign Application Priority Data
Sep. 28, 2001 (NZ) 514547

(51) Int. Cl. A01H 1/00; C12Q 1/68; C07H 21/04; C12N 9/32; C12N 15/82; C12N 5/04

(52) U.S. Cl. 800/284; 435/6; 435/468; 435/419; 435/204; 536/23.2

(57) ABSTRACT

The present invention relates to isolated polypeptides having alpha-amylase activity and/or starch binding activity and/or plasmid targeting signals and to isolated polynucleotides encoding the polypeptides. The invention also relates to DNA constructs, vectors and host cells incorporating the polynucleotide sequences, methods for modulating starch content in plants, particularly plastids, as well as modifying plastid specific starch. The applicants have identified a class of alpha-amylases with plastid targeting signals and starch binding activity.

### FIELD OF THE INVENTION

The present invention relates to isolated polypeptides having alpha-amylase (α-amylase) activity and/or starch binding activity and/or plastid targeting signals and to isolated polynucleotides encoding the polypeptides. The invention also relates to genetic constructs, vectors and host cells incorporating the polynucleotide sequences, methods for modulating starch content in plants, particularly plastids, as well as modifying plastid specific starch.

### BACKGROUND

Starch is a vital carbon storage molecule for most plants. It is insoluble and stored within the plastids of plant cells. Starch can be separated into one of two basic groups by its function. The first, transitory (or diurnal) starch is produced in photosynthetic tissues during the day, and at night it is broken down into sugars and exported to other parts of the plant. The recipient tissues can feed the sugars directly into catabolic pathways, or they can re-synthesise starch for longer-term storage. This storage starch forms the second group and is produced in a variety of organs, including roots, tubers, bark/wood and developing fruit.

α-Amylase (E.C. 3.2.1.1) is a starch endo-hydrolase, which cleaves α-1,4-glucan bonds within starch molecules. It is made up of three domains: domain A folds into a (β/α)₈ barrel, and contains the catalytic residues of the enzyme; domain B is a large loop that protrudes from between the third β-strand and the third α-helix of domain A; domain C is located at the C-terminal end of the (β/α)₈ barrel, and is made up of β-strands. The functions of domains B and C are largely unknown, although domain B has been shown to influence several isozyme-specific properties of barley α-amylases, including substrate binding, catalysis, and stability at low pH.

Much of the work on plant α-amylases has focussed on enzymes from monocotyledonous plants, particularly the cereals barley and rice. α-Amylase plays a vital role in the germination of cereal grains; it is secreted from the aleurone layer into the endosperm where it initiates starch hydrolysis. However α-amylases are not limited to tissues of germinating seeds. α-Amylase activity has also been detected in water stressed leaves of barley, and in cultured rice cells, localised to the cell walls and amyloplasts. Molecular studies of α-amylase genes in monocots have revealed a large but well conserved family, often represented by multiple genes in each plant, e.g. at least 10 genes in rice. The proteins encoded by these genes all have N-terminal signal peptides, which would channel them into the cellular secretory pathway. No monocot α-amylases targeted to intracellular locations have been identified to date.

Dicot α-amylases have received very little attention compared to the monocot enzymes. To date, only a handful of α-amylase genes have been identified in dicots, predominantly in germinating cotyledons, most with high homology to previously characterised monocot genes. As for monocots, most dicot α-amylases possess putative signal peptides.

For the known dicotyledenous α-amylases, specific function and sub-cellular localisation has yet to be elucidated. Given the importance of starch storage in plants and plastids in particular, it would be desirable to identify α-amylases implicated to function in plastids.

It would also be desirable to identify polypeptide signal sequences directing such plastid-localised α-amylases to plastids. The nucleotide sequences encoding such plastid targeting signals could be utilised to direct chimeric proteins, containing the plastid targeting signals, to the plastids of transgenic plants. This compartmentalisation could avoid any toxic effects of expressed protein, on the contents of the cell outside the plastids. Additionally plastid localisation could avoid the deleterious effects of cytoplasmic factors on the expressed protein. It would also be advantageous to target polypeptides intended to interact with plastid factors, to plastids.

Further, it would be desirable to identify polypeptide sequences implicated in starch binding in plants. Chimeric recombinant proteins including such starch binding sequences could be produced. Such recombinant proteins could have high value in industrial processes. For example they could be used to modify industrial materials that use starch or starch derivatives as their polymeric base, such as biodegradable films.

The applicants have now identified and isolated from apple a polynucleotide encoding a novel α-amylase polypeptide which contains a plastid targeting signal. It is broadly towards this polynucleotide, to its homologs and to the modulation of its expression/function within plants that the present invention is directed.

The applicants have also identified, for the first time, in plant α-amylases, peptide motifs implicated in plastid targeting of α-amylases. The invention also relates utilisation of this sequence and its homologues, to facilitate plastid targeting of chimeric protein in transgenic plants.

The applicants have further identified for the first time in plant α-amylases, polypeptide motifs implicated in starch binding of α-amylases. The invention further relates to utilization of these peptide sequences, to produce recombinant chimeric proteins with starch binding properties.

### SUMMARY OF THE INVENTION

In a first aspect, the present invention provides an isolated polypeptide, which encodes a plastid α-amylase.

In a further aspect, the present invention provides an isolated polypeptide having the Mdamy10 amino acid sequence of SEQ ID NO:2 or a functionally equivalent variant thereof. The invention also provides the isolated mature polypeptide.

In a further aspect, the present invention provides an isolated polynucleotide which encodes a plastid targeted α-amylase polypeptide having the Mdamy10 amino acid sequence of SEQ ID NO:2 or a functionally equivalent variant thereof.

Preferably, said polynucleotides comprise part or all of the nucleotide sequence of SEQ ID NO:1.

In one embodiment, there is provided a polynucleotide sequence of the invention, which encodes a novel N-terminal domain of an α-amylase, and comprises the sequence of SEQ ID NO:3.

The invention also provides a polynucleotide sequence of the invention which encodes the N-terminal domain comprising of SEQ ID NO:4 or a functionally equivalent variant thereof.

The invention also provides a polynucleotide sequence which encodes a novel starch binding domain polypeptide of SEQ ID NO:5 or a functionally equivalent variant thereof.

The invention also provides a polynucleotide sequence of SEQ ID NO:6.

Also provided is a polynucleotide sequence which encodes a plastid targeting polypeptide selected from SEQ ID NO:7, SEQ ID NO:8, residues 1-70 of SEQ ID NO:10 and residues 1-53 of SEQ ID NO:53 or a functionally equivalent variant thereof.

The invention also provides a polynucleotide sequence coding for a polypeptide which comprises at least one repeat, preferably two, of the defined polypeptide motif pair. The motif pair can be defined by aligning of the N-terminal half of the family three α-amylases, namely Mdamy10, Mdamy11, Atamy3, Osamy10 (from rice, Oryza sativa), and Fragment 1 from kiwifruit (Actinidia chinensis)(FIG. 8):
Motif 1: yHWGV[X]₇₋₁₀W(D/E)(Q/I)P(P)[X]₃₋₄P(P)[X]₈A(I/L)XTXL
Motif 2: FV(F/L/V)K[X]₂E[X]₂₋₃W[X]₄₋₆GXDF
   or functionally equivalent variants thereof.

(In this notation, capital letters represent conserved amino acids, whilst letters in parentheses represent partly conserved amino acids; y represents a hydrophobic residue; X represents any amino acid; and [α]₄₋₆ represents a run of 4 to 6 unspecified amino acids).

The polynucleotide is preferably DNA.

The motifs can be more loosely defined if sequences from other putative starch binding proteins are included. The sequences added were R1 protein from potato, its homolog from Arabidopsis, Sex1, and a putative starch branching enzyme (SBE-like) from Arabidopsis (FIG. 9). The resulting motifs are:
Motif 1: yHW(G/A)y[X]₆₋₉WXXP[X]₃₋₅PXX(T/S)
Motif 2: F(V/L)y[X]₅₋₈W[X]₆₋₈(D/N)F
   or functionally equivalent variants thereof. The same notation is used as above.

The polynucleotide is preferably DNA.

The invention further provides a genetic construct, which includes a polynucleotide as defined above.

More particularly, the invention provides a genetic construct comprising in the 5′-3′ direction:
(a) a promoter sequence;
(b) an open reading frame polynucleotide coding for a polypeptide of the invention or a functionally equivalent variant thereof; and
(c) a termination sequence.

Preferably, the polypeptide comprises an amino acid sequence selected from SEQ ID NO:2, SEQ ID NO:4, SEQ ID NO:5, SEQ ID NO:7 and SEQ ID NO:8.

In one embodiment, the open reading frame is in a sense orientation.

In an alternate embodiment, the open reading frame is in an anti-sense orientation.

In still a further embodiment, the invention provides a genetic construct comprising, in the 5′-3′ direction:
(a) a promoter sequence;
(b) a non-coding region of, a polynucleotide which encodes a polypeptide of the invention or a functionally equivalent variant thereof; and
(c) a termination sequence.

Preferably, the polypeptide has an amino acid sequence selected from SEQ ID NO:2, SEQ ID NO:4, SEQ ID NO:5, SEQ ID NO:7 and SEQ ID NO:8.

Once again, the non-coding region can be in a sense or anti-sense orientation.

In yet a further embodiment, the invention provides a genetic construct comprising, in the 5′-3′ direction:
(a) a promoter sequence;
(b) a polynucleotide comprising a polynucleotide sequence complementary to at least part of a sequence coding for a polypeptide of the invention, or a functionally equivalent variant of either; and
(c) a termination sequence.

In one embodiment, the polynucleotide of the genetic construct in (b), includes an inverted repeat, such that a hairpin structure can be formed from the transcript.

Preferably, in each embodiment, the genetic construct further includes a marker for identification of transformed cells.

In a further aspect, the invention provides a host cell, which includes a genetic construct as defined above.

In still a further aspect, the invention provides a transgenic plant cell which includes a genetic construct as defined above, as well as a transgenic plant comprising such cells.

In a yet further aspect, the invention provides a plant which has been genetically modified to alter the expression and/or activity of a polypeptide of the sequence in SEQ ID NO:2 or a functionally equivalent variant thereof.

In a further aspect, the invention provides a plant which contains a genetic construct comprising a polynucleotide encoding a polypeptide having the Mdamy10 SEQ ID NO: 2 or SEQ ID NO:4 or a functionally equivalent variant thereof and in which expression and/or activity of said polypeptide within said plant has been disrupted.

Such a plant will accumulate starch in the plastid and in the organ where starch is stored (e.g. leaves, tubers, roots, bark/wood, fruit) moreover the starch structure and composition will be altered. In addition primary metabolism will be altered in the transformed tissue and the plant will have altered carbohydrate transport between the various organs.

In a further aspect, the invention provides a plant which has been genetically modified such that it does not functionally express a polypeptide selected from SEQ ID NO:4 and SEQ ID NO:5.

In such a plant starch binding capacity of the polypeptide is reduced which means proteins affected are unable to function in starch metabolism.

In one form, functional expression of said polypeptide encoded by the polynucleotide is disrupted directly.

In another form, functional expression of said polypeptide encoded by polynucleotide is disrupted indirectly, such as through disrupting functional expression of the polypeptide encoded by said polynucleotide.

As used herein “plant” means gymonosperm, angiosperm, monocotyledenous or dicotyledenous plants, plant parts such as leaves, roots, flowers, fruit, bark/wood, tubers, cuttings, seeds, tissue cultures, cell cultures and plant cells but is not limited thereto.

As used herein, “functional expression” of said polypeptide refers to the amount of the polypeptide which is expressed and functional within the plant. For example, a plant which does not functionally express a polypeptide can mean either that there is no expression of that polypeptide at all, or that the polypeptide is expressed but no longer performs its previous function.

Disruption of expression and/or activity may be by mutation (such as frameshift, deletion, insertion or knockout mutations) of the polynucleotide itself or of its regulatory elements, or by down-regulation (such as antisense or co-suppression) or any other method known to those skilled in the art by which aberrant or reduced expression of the gene may be achieved. Disruption may therefore be specifically caused by down-regulation of expression of Mdamy10.

It is appreciated that experiments designed to decrease expression levels of polypeptides of the invention, may result in a range of expression levels in different transgenic plants. The different levels of expression will result in different levels of α-amylase activity, any of which may provide useful plants.

The invention also provides a method for modulating the starch content of a plant, the method comprising increasing or decreasing expression and/or activity of the polypeptide selected from SEQ ID NO:2, SEQ ID NO:4, SEQ ID NO:5, SEQ ID NO:7 and SEQ ID NO:8 by genetic modification to alter the expression of a gene encoding a plastid targeted α-amylase.

Starch metabolism may be altered by introducing into a plant a genetic construct of the invention and expressing the polypeptide in the plant.

The invention also broadly relates to the use of the polynucleotides and polypeptides of the invention to modulate starch content of organisms including plants.

Also specifically contemplated is the use of the N-terminal and starch binding domain and/or specific starch binding motifs in the production of recombinantly expressed chimeric polypeptides containing such starch binding domains and or motifs. Such recombinant chimeric polypeptides could possess the ability to bind starch.

Also specifically contemplated is the use of the polynucleotide sequences encoding the plastid targeting motifs of the invention, to prepare chimeric genetic constructs, which direct the targeting of transgenically expressed polypeptides to the plastids of transgenic plants.

While the invention is broadly defined as above, those persons skilled in the art will appreciate that it is not limited thereto and that it also includes embodiments of which the following description provides examples.

### BRIEF DESCRIPTION OF T HE DRAWINGS

In addition, the present invention will be better understood from reference to the accompanying drawings in which:

FIG. 1 shows a comparative alignment of Family three α-amylase sequences with apple α-amylase 8 (Mdamy8). Mdamy8 is a cytosol-targeted α-amylase. Each bar is shaded to distinguish the four protein domains encoded by the sequences: A, B and C represent the structural domains A, B and C, which are found throughout all α-amylases; Novel domain refers to the N-terminal domain found only in Family three α-amylases. Mdamy8, Atamy3, Mdamy10, and Osamy10 all represent full-length sequences. Mdamy11 and sequences from Kiwifruit, Blueberry, Coffee, Cotton, and Rose, all represent partial sequences.

FIG. 2 shows the nucleotide sequence of Mdamy10. The nucleotides encoding the plastid targeting peptide (nucleotides 55 to 237) are shown in bold, while the nucleotides encoding the novel starch binding domain are underlined (238 to 1503).

FIG. 3 shows the amino acid sequence of Mdamy10. The plastid targeting peptide (amino acids 1 to 61) is shown in bold, the novel starch binding domain is underlined (62 to 483).

FIG. 4 shows the nucleotide sequence of Atamy3. The nucleotides encoding the plastid targeting peptide (nucleotides 1 to 165) are shown in bold, while the nucleotides encoding the novel starch binding domain are underlined (166 to 1410).

FIG. 5 shows the amino acid sequence of Atamy3. The plastid targeting peptide (amino acids 1 to 55) is shown in bold, the novel starch binding domain is underlined (56 to 470).

FIG. 6 shows the nucleotide sequence of Osamy10. The nucleotides encoding the plastid targeting peptide (nucleotides 1 to 159) are shown in bold, while the nucleotides encoding the novel starch binding domain are underlined (160 to 1371).

FIG. 7 shows the amino acid sequence of Osamy10. The plastid targeting peptide (amino acids 1 to 53) is shown in bold, the novel starch binding domain is underlined (54 to 457).

FIG. 8 shows an alignment of polypeptide sequences from the starch binding domains of Family three α-amylases. Sequence titles are shown to the left of the sequences themselves. The kiwifruit sequence refers to sequence 1 (SEQ ID NO 21). The letter ‘a’ indicates that the sequence is the first (closest to the N-terminus) repeat of a motif pair within the sequence; ‘b’ indicates the second (closest to the C-terminus) repeat of a motif pair. Shading indicates sequence conservation: Black=fully conserved, white letters on gray background=highly conserved, black letters on gray background=moderately conserved.

FIG. 9 shows an alignment of polypeptide sequences from the starch binding domains of Family three α-amylases, plus R1 protein from potato (Genbank-CAA70725), its homolog from Arabidopsis, Sex1 (Genbank-AAG47821), and a putative starch branching enzyme (SBE-like) from Arabidopsis (Genbank-BAB02827). Sequence titles are shown to the left of the sequences themselves. The kiwifruit sequence refers to sequence 1 (SEQ ID NO 21). The letter ‘a’ indicates that the sequence is the first (closest to the N-terminus) repeat of a motif pair within the sequence; ‘b’ indicates the second (closest to the C-terminus) repeat of a motif pair. Shading indicates sequence conservation: Black=fully conserved, white letters on gray background=highly conserved, black letters on gray background=moderately conserved.

FIG. 10 shows results of PCR with primers NewUNIf2 and NewUNIr2 on genomic DNA from a number of plant species: Lane 1; pine. Lane 2; potato. Lane 3; onion. Lane 4; rose. Lane 5; olive. Lane 6; coffee. Lane 7; banana. Lane 8; mango. Lane 9; cotton. Lane 10; 1 kb+DNA ladder (Bio-Rad Laboratories). Indicative sizes are shown to the right. Samples were electrophoresed through a 0.8% agarose gel and stained with ethidium bromide.

FIG. 11 shows fluorescence microscopy images of GFP fusion genetic constructs expressed in N. benthamiana epidermal cells. Top left, GFP alone (pDS-GFP-ART27), using GFP plant filter set; bar represents 50 μm. Top right, cTP-GFP (pcTP-ART27), using UV filter set; bar represents 50 μm. Bottom left, cTP-GFP, using GFP plant filter set; bar represents 50 μm. Bottom right, cTP-GFP, using GFP plant filter set; bar represents 10 μm.

FIG. 12 Shows SDS-PAGE (A) and western blot (B) of protein from IPTG induced E. coli containing pET-30a-based GFP constucts. The western was probed with anti-GFP antibodies. Lanes 1-4 are insoluble protein fractions. Lanes 6-9 are soluble protein fractions. Lanes 1 and 6—pET-30a; Lanes 2 and 7—pGFP-ET-30b; Lanes 3 and 8—pNterm-ET-30b; Lanes 4 and 9—pSBD-ET-30a; Lane 5—Prestained Broad Range SDS-PAGE Standard (Bio-Rad)—sizes shown at right of figure.

FIG. 13 shows protein samples on amylopectin-containing gels that have been stained with iodine. Gel A: Lane 1 contains crude protein extract from E. coli expressing pET-30a. Lane 2 contains crude protein extract from E. coli expressing pAmy10-ET-30a. Lane 3 contains crude pET-30a extract desalted in binding buffer. Lane 4 contains crude pAmy10-ET-30a extract desalted in binding buffer. Gel B: Lanes 5, 7 and 9 contain nickel-purified pET-30a protein, from consecutive 2.5 mL fractions. Lanes 6, 8 and 10 contain nickel-purified pAmy10-ET-30a protein, from consecutive 2.5 mL fractions. Lane 11 contains crude protein extract from E. coli expressing pAmy10-ET-30a.

FIG. 14 shows a starch transfer gel that has been stained with iodine. Lane 1 contains crude protein extract from E. coli expressing pET-30a only. Lane 2; crude protein extract from E. coli expressing pAmy10-ET-30a. Lane 3; protein extracted from apple leaves. Lane 4; protein extracted from Arabidopsis leaves. Lane 5; crude protein extract from E. coli expressing pAmy10-ET-30a.

FIG. 15 shows SDS-PAGE (A) and western blot (B) of protein from IPTG induced E. coli containing pET-30a and pAmy10-ET-30a plasmids. The western was probed with anti-His₆ antibodies. Lane 1—crude pET-30a; Lane 2—crude pAmy10-ET-30a; Lane 3—desalted pET-30a; Lane 4—desalted pAmy10-ET-30a; Lane 5—Pre-stained Broad Range SDS-PAGE Standard (Bio-Rad)—sizes shown at right of figure; Lane 6—pAmy10-ET-30a, purification fraction 1; Lane 7—pET-30a, purification fraction 2; Lane 8—pAmy10-ET-30a, purification fraction 2; Lane 9—pAmy10-ET-30a, purification fraction 3 (part of the lane was removed prior to semi-dry blotting, and so does not appear complete on the blot (B)).

FIG. 16 shows RT-PCR of Arabidopsis α-amylase transcripts from ten different tissues.

PCR amplification was carried out for thirty cycles, using primers designed to amplify part of each Arabidopsis α-amylase (Atamy1, 2 or 3). Tissues: 1: Imbibed seeds, 2: Seeds with emerging cotyledons, 3: Cotyledons only from (2), 4: Whole seedling (2 leaves), 5: Leaves only from (4), 6: Roots, 7: Full rosette plant, 8: Leaves from growing stem, 9: Young seed pods, 10: Senescing plant (minus seed pods).

### DESCRIPTION OF THE INVENTION

As broadly outlined above, the applicants have identified novel α-amylases targeted to the plastid of a plant. In a preferred embodiment the plant is a fruiting plant. Polynucleotides coding for the novel polypeptides are also provided. The specific polypeptides and polynucleotides are from the plants Malus x domestica, Arabidopsis thaliana and Oryza sativa.

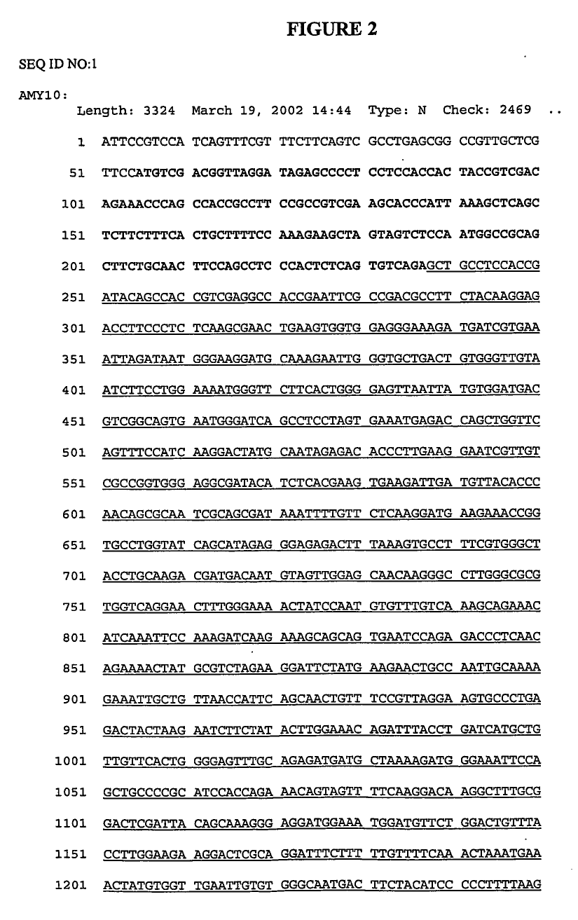

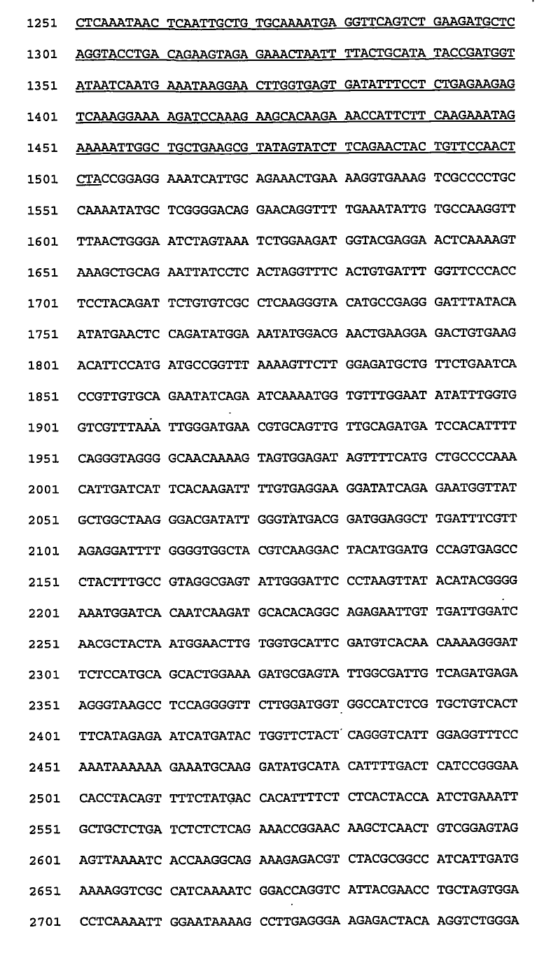

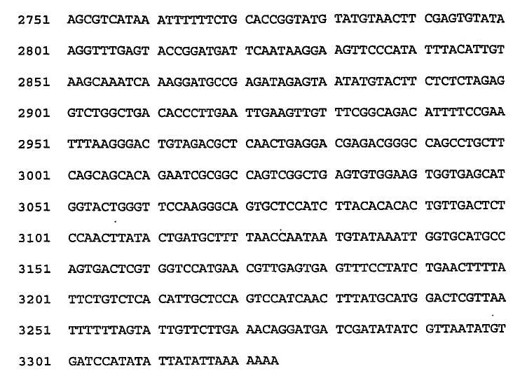

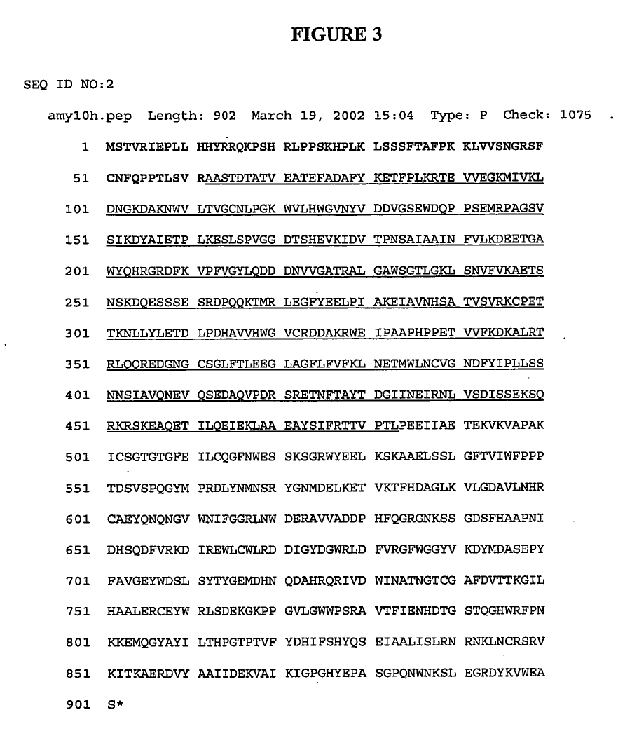

The amino acid sequence of one polypeptide, Mdamy10, and that of the polynucleotide sequence encoding it are given in FIGS. 3 and 2 respectively. It will however be appreciated that the invention is not restricted only to the polypeptide/polynucleotide having the specific amino acid/nucleotide sequence given in FIGS. 3 and 2. Instead, the invention also extends to functionally equivalent variants of the polypeptide/polynucleotide of FIGS. 3 and 2.

The term “polynucleotide(s)” as used herein means a single or double-stranded polymer of deoxyribonucleotide or ribonucleotide bases and includes DNA and corresponding RNA molecules, including hnRNA and mRNA molecules, both sense and anti-sense strands, and comprehends cDNA, genomic DNA and recombinant DNA, as well as wholly or partially synthesized polynucleotides. An hnRNA molecule contains introns and corresponds to a DNA molecule in a generally one-to-one manner. An mRNA molecule corresponds to an hnRNA and DNA molecule from which the introns have been excised. A polynucleotide may consist of an entire gene, or any portion thereof. Operable anti-sense polynucleotides may comprise a fragment of the corresponding polynucleotide, and the definition of “polynucleotide” therefore includes all such operable anti-sense fragments.

The term ‘polypeptide(s)’ as used herein includes peptides, polypeptides and proteins.

The phrase “functionally equivalent variants” recognises that it is possible to vary the amino acid/nucleotide sequence of a polypeptide while retaining substantially equivalent functionality. For example, a polypeptide can be considered a functional equivalent of another polypeptide for a specific function if the equivalent polypeptide is immunologically cross-reactive with and has at least substantially the same function as the original polypeptide. The equivalent can be, for example, a fragment of the polypeptide, a fusion of the polypeptide with another polypeptide or carrier, or a fusion of a fragment with additional amino acids.

Variant polynucleotide sequences also include equivalent sequences, which vary in size, composition, position and number of introns, as well as size and composition of untranslated terminal regions.

Functionally equivalent polynucleotides are those encoding functionally equivalent polypeptides.

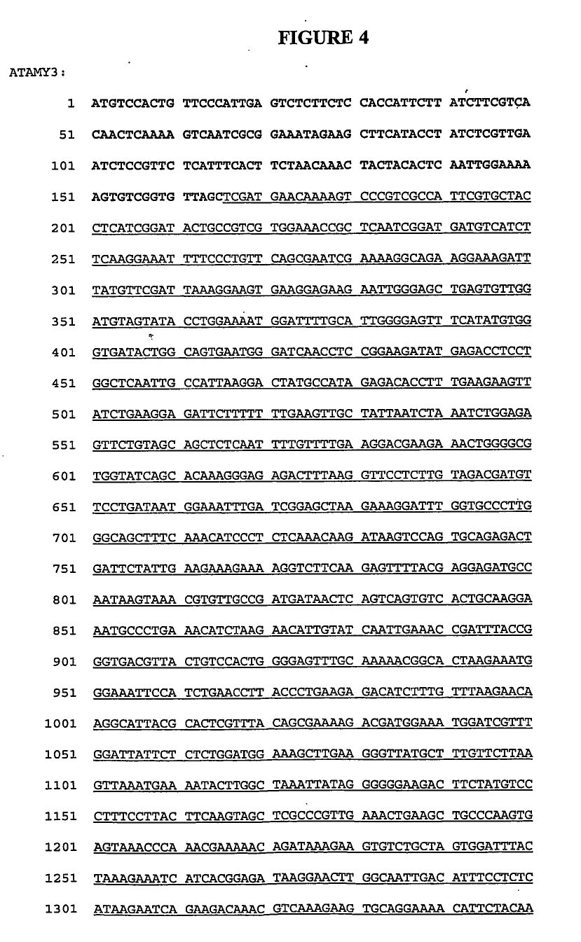

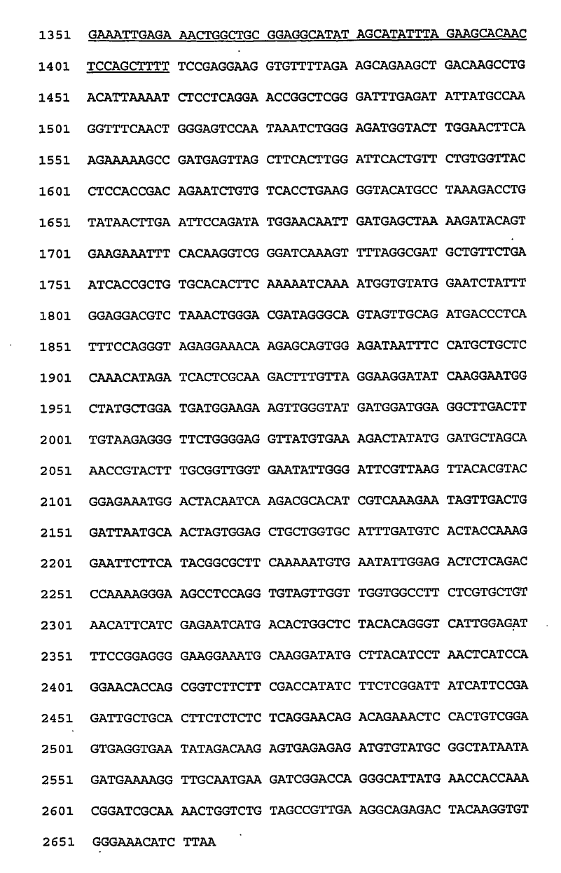

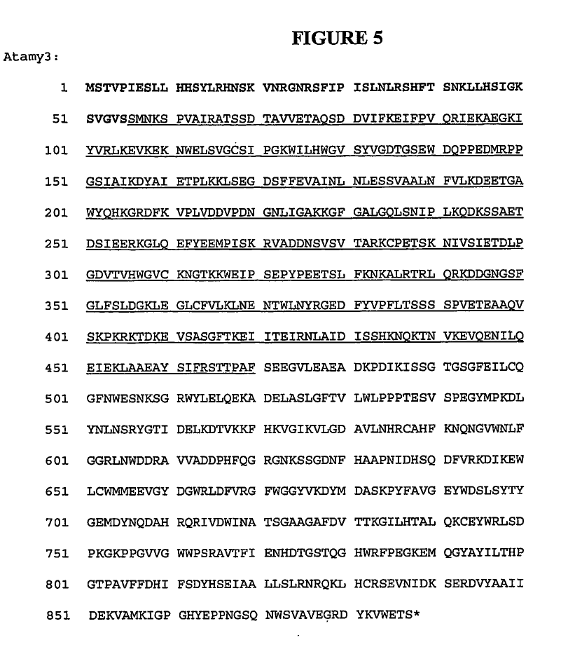

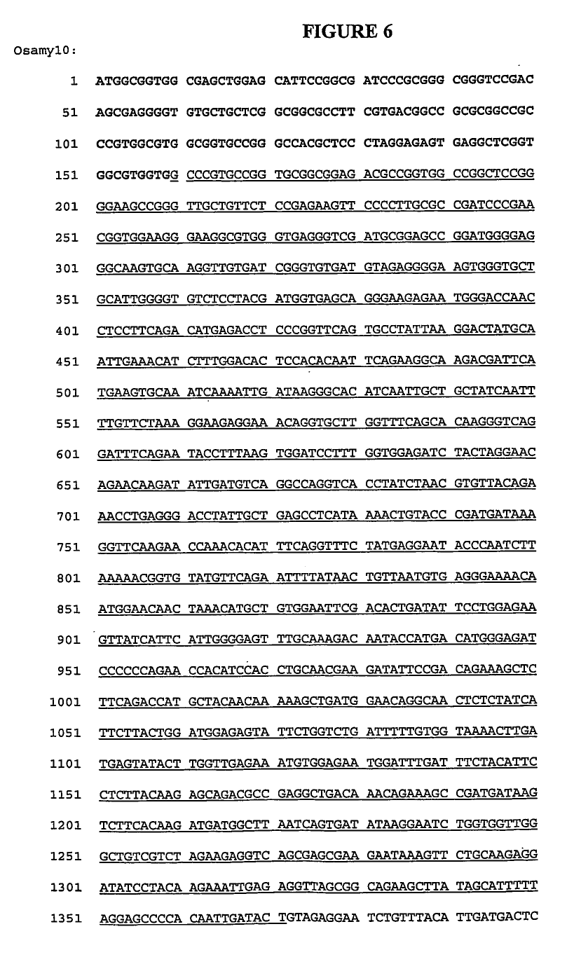

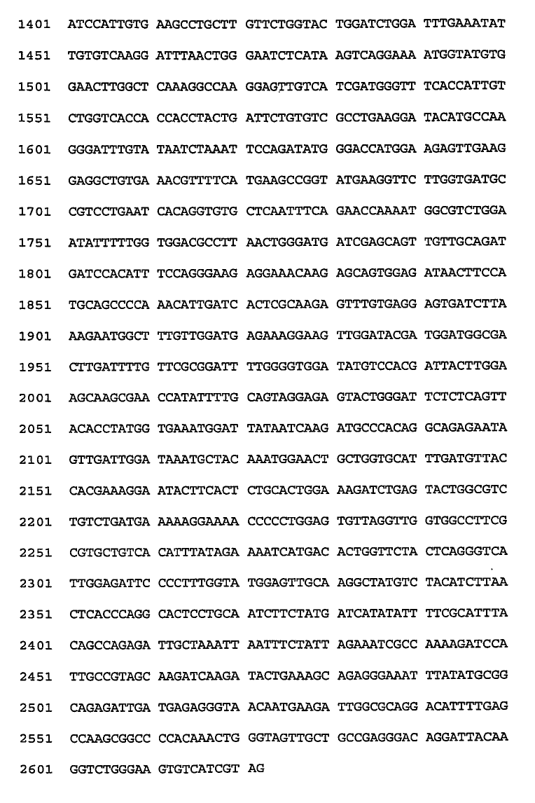

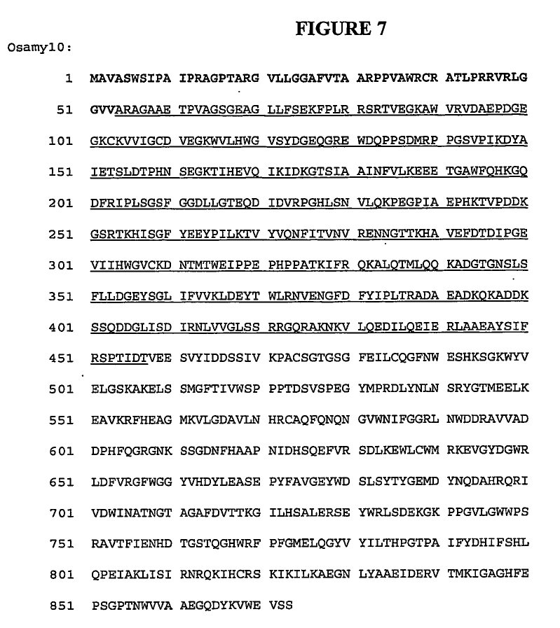

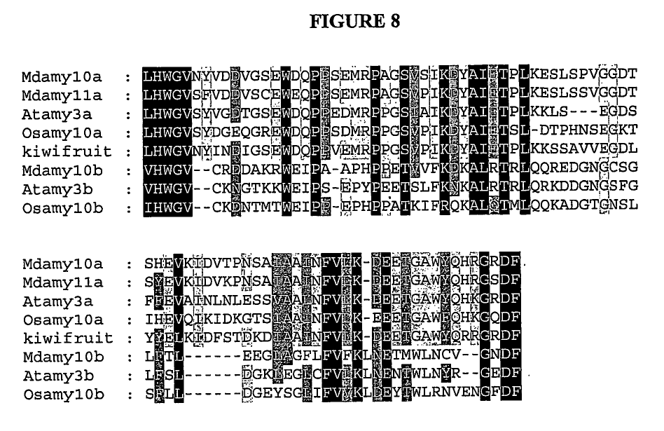

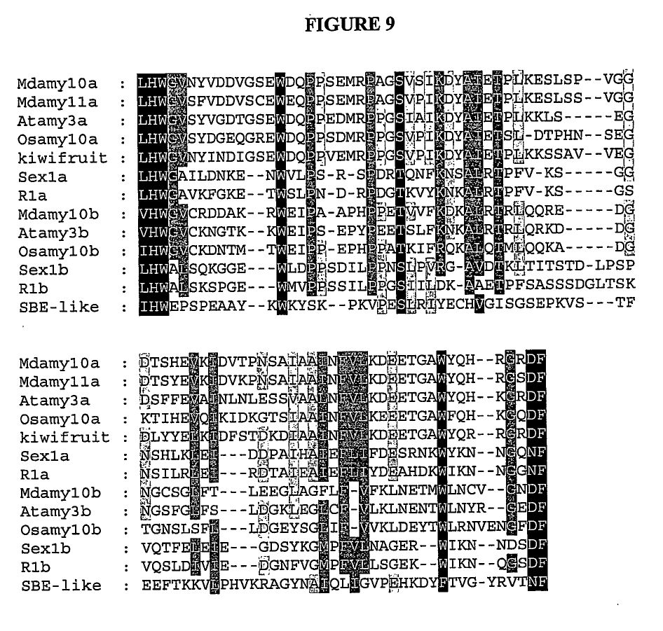

The polynucleotide sequence encoding important N-terminal polypeptide region of Mdamy 10 is shown in FIG. 2. The corresponding N-terminal amino acid sequence from Mdamy10 is shown in FIG. 3. The large N-terminal extension (approximately 460-480 amino acids) was originally found in Mdamy10 and a homologue from Arabidopsis, Atamy3 (GenBank Accession No. A7050398; FIGS. 4 and 5). A further plastid-targeted sequence was identified by the applicants from apple (Mdamy11), and then later from rice (Osamy10; FIGS. 6 and 7). These N-terminal extensions include plastid-targeting peptides of 61 amino acids in Mdamy10 (SEQ ID NO:2 and SEQ ID NO:7), 70 amino acids in Mdamy11 (SEQ ID NO:10), 55 amino acids in Atamy3 (SEQ ID NO:17 and SEQ ID NO:8) and 53 amino acids in Osamy10 (SEQ ID NO:53).

The N-terminal regions have also been found to include a potential starch binding region (SEQ ID NO:5), and potential specific starch binding motifs as set out above. The remaining C-terminal regions (the last 420 amino acids in Mdamy10, the last 416 in Atamy3, and Osamy10) include the α-amylase region. Mdamy11 sequences contain most of a starch binding domain, in amino acids 71 to 243 of the 5′ polypeptide fragment (SEQ ID NO:10), which includes a complete pair of starch binding motifs, and amino acids 1 to 107 of the central fragment (SEQ ID NO:12), which includes part of a starch binding motif 2. Amino acids 108 to 172 of the central fragment and 1 to 142 of the 3′ fragment (SEQ ID NO:14) all constitute part of an α-amylase region.

The applicants have also determined that Mdamy10, Mdamy11, Atamy3, and Osamy10 form a distinct family of α-amylases and have named these Family three α-amylases. This was determined not only by their plastid targeting but also based on the number and distribution of their introns. Atamy3 and Osamy10 have 12 introns that interrupt their coding sequences; their full genomic sequences, including introns, are SEQ ID NO:55 and SEQ ID NO:54, respectively. Six of their introns are within the α-amylase coding region of the gene (the 3′ half) as shown in Table 1 below.

TABLE 1

| | 1^{a} | 2 | 3 | 4 | 5 | 6 | 7 | 8 |
|---|---|---|---|---|---|---|---|---|
| | 96-0 | 138-2 | 157-0 | 195-0 | 231-1 | 342-0 | 501-0 | 544-1 |
| Hvamy2-2 | n/a | n/a | n/a | n/a | n/a | n/a | + | − |
| AmyVm1 | n/a | n/a | n/a | n/a | n/a | n/a | + | + |
| Mdamy10 | ? | ? | ? | ? | ? | ? | ? | ? |
| Mdamy11 | ? | ? | + | ? | ? | ? | ? | ? |
| Atamy3 | + | + | + | + | + | + | − | − |
| Osamy10 | + | + | + | + | + | + | − | − |

| | 9 | 10 | 11 | 12 | 13 | 14 | 15 |
|---|---|---|---|---|---|---|---|
| | 556-2 | 620-0 | 655-2 | 729-0 | 780-0 | 806-0 | 836-0 |
| Hvamy2-2 | − | − | − | − | − | + | − |
| AmyVm1 | − | − | − | − | − | + | − |
| Mdamy10 | ? | ? | + | + | + | ? | ? |
| Mdamy11 | ? | ? | + | + | + | ? | ? |
| Atamy3 | + | + | + | + | + | − | + |
| Osamy10 | + | + | + | + | + | − | + |

The distribution of introns in Family one and Family three α-amylase genes. The codon numbers correspond to and are equivalent to the amino acid sequence of Atamy3 (FIG. 5). The presence of an intron is represented by a + sign and its absence by a − sign. The regions of Mdamy10 and Mdamy11 for which there is no genomic DNA sequence is marked ?. The region from amino acids 1-470 of Atamy3 has no equivalent in Hvamy2-2 and amyVm1, these regions have been marked n/a. The applicants have defined a total of 15 distinct intron positions amongst the Family one and Family three genes, they are numbered from the 5′ of the Atamy3 gene sequence and defined by codon number and phase. For introns separating triplets after the first base, the codon number is followed by −1. For introns separating triplets after the second base, the codon number is followed by −2. For introns falling between triplets the number of the codon located 3′ of the intron is given, followed by −0. GenBank Accession numbers: Hvamy2-2 (AAA98790), AmyVm1 (CAA37217).

All three α-amylases have short 5′ untranslated regions (UTRs) between 35 and 54 base pairs. However, Mdamy10 has a very long 3′ UTR, of up to 557 base pairs.

Table 2 provides information on the characterisation of the UTRs of Mdamy10, Mdamy11 and Atamy3.

TABLE 2

| | 5′ UTR Length | 3′ UTR Length | No of polyadenylation |
|---|---|---|---|
| | (Max/Min) (bp) | (Max/Min) (bp) | sites detected |
| Mdamy10 | 54 | 557/428 | 4 |
| Mdamy11 | 35 | 98^{a} | 1 |
| Atamy3 | 46 | 229 | Unknown |

^{a}Refers to the 3′ UTR of the mis-spliced, truncated Mdamy11 transcript.

The unique intron structure which characterises this α-amylase family is also believed to affect expression of the α-amylases.

It will be understood that a variety of substitutions of amino acids is possible while preserving the structure responsible for activity of the polypeptides. Conservative substitutions are described in the patent literature, as for example, in U.S. Pat. No. 5,264,558 or 5,487,983. It is thus expected, for example, that interchange among non-polar aliphatic neutral amino acids, glycine, alanine, proline, valine and isoleucine, would be possible. Likewise, substitutions among the polar aliphatic neutral amino acids, serine, threonine, methionine, asparagine and glutamine could be made. Substitutions among the charged acidic amino acids, aspartic acid and glutamic acid, could probably be made, as could substitutions among the charged basic amino acids, lysine and arginine. Substitutions among the aromatic amino acids, including phenylalanine, histidine, tryptophan and tyrosine are also possible. Such substitutions and interchanges are well known to those skilled in the art.

Equally, nucleotide sequences encoding a particular product can vary significantly simply due to the degeneracy of the nucleic acid code.

A polynucleotide or polypeptide sequence may be aligned, and percentage of identical nucleotides in a specified region may be determined against another sequence, using computer algorithms that are publicly available. Two exemplary algorithms for aligning and identifying the similarity of polynucleotide sequences are the BLASTN and FASTA algorithms. The similarity of polypeptide sequences may be examined using the BLASTP algorithm. Both the BLASTN and BLASTP software are available on the NCBI anonymous FTP server (ftp://ncbi.nlm.nih.gov) under /blast/executables/. The BLASTN algorithm version 2.0.4 [Feb. 24, 1998], set to the default parameters described in the documentation of variants according to the present invention. The use of the BLAST family of algorithms, including BLASTN and BLASTP, is described at NCBI's website at URL http://www.ncbi.nlm.nih.gov/BLAST/newblast.html and in the publication of Altschul, Stephen F., et al. (1997). The computer algorithm FASTA is available on the Internet at the ftp site ftp://ftp.virginia.edu/pub/fasta/. Version 2.0u4, February 1996, set to the default parameters described in the documentation and distributed with the algorithm, is also preferred for use in the determination of variants according to the present invention. The use of the FASTA algorithm is described in (Pearson and Lipman 1988, Pearson 1990).

The following running parameters are preferred for determination of alignments and similarities using BLASTN that contribute to E values (as discussed below) and percentage identity: Unix running command: blastall -p blastn -d embldb -e 10-G 1-E 1-r 2-v 50-b 50
-I queryseq -o results; and parameter default values:
-p Program Name [String]
-d Database [String]
-e Expectation value (E) [Real]
-G Cost to open a gap (zero invokes default behaviour) [Integer]
-E Cost to extend a cap (zero invokes default behaviour) [Integer]
-r Reward for a nucleotide match (blastn only) [Integer]
-v Number of one-line descriptions (V) [Integer]
-b Number of alignments to show (B) [Integer]
-i Query File [File In]
-o BLAST report Output File [File Out] Optional

For BLASTP the following running parameters are preferred: blastall -p blastp -d swissprotdb -e 10-G 1-E 1-v 50-b 50-I queryseq -o results
-p Program Name [String]
-d Database [String]
-e Expectation value (E) [Real]
-G Cost to open a gap (zero invokes default behaviour) [Integer]
-E Cost to extend a cap (zero invokes default behaviour) [Integer]
-v Number of one-line descriptions (v) [Integer]
-b Number of alignments to show (b) [Integer]
-i Query File [File In]
-o BLAST report Output File [File Out] Optional

The “hits” to one or more database sequences by a queried sequence produced by BLASTN, BLASTP, FASTA, or a similar algorithm, align and identify similar portions of sequences. The hits are arranged in order of the degree of similarity and the length of sequence overlap. Hits to a database sequence generally represent an overlap over only a fraction of the sequence length of the queried sequence.

The BLASTN and FASTA algorithms also produce “Expect” or E values for alignments. The E value indicates the number of hits one can “expect” to see over a certain number of contiguous sequences by chance when searching a database of a certain size. The Expect value is used as a significance threshold for determining whether the hit to a database, such as the preferred EMBL database, indicates true similarity. For example, an E value of 0.1 assigned to a hit is interpreted as meaning that in a database of the size of the EMBL database, one might expect to see 0.1 matches over the aligned portion of the sequence with a similar score simply by chance. By this criterion, the aligned and matched portions of the sequences then have a 90% probability of being the same. For sequences having an E value of 0.01 or less over aligned and matched portions, the probability of finding a match by chance in the EMBL database is 1% or less using the BLASTN or FASTA algorithm.

According to one embodiment, “variant” polynucleotides, with reference to each of the polynucleotides of the present invention, preferably comprise sequences having the same number or fewer nucleic acids than each of the polynucleotides of the present invention and producing an E value of 0.01 or less when compared to the polynucleotide of the present invention. That is, a variant polynucleotide is any sequence that has at least a 99% probability of being the same as the polynucleotide of the present invention, measured as having an E value of 0.01 or less using the BLASTN or FASTA algorithms set at the parameters discussed above.

Variant polynucleotide sequences will generally hybridize to the recited polynucleotide sequence under stringent conditions. As used herein, “stringent conditions” refers to prewashing in a solution of 6×SSC, 0.2% SDS; hybridizing at 65° C., 6×SSC, 0.2% SDS overnight; followed by two washes of 30 minutes each in 1×SSC, 0.1% SDS at 65° C. and two washes of 30 minutes each in 0.2×SSC, 0.1% SDS at 65° C. The variant polynucleotide sequences of the invention are at least 50 nucleotides in length.

Variant polynucleotides also include sequences which encode a polypeptide that has a sequence identity of at least 60%, generally 70%, preferably 80%, more preferably 90%, even more preferably 95%, very preferably 98% and most preferably 99% or more to the nucleotide of its respective native nucleotide sequence given in the sequence listing herein.

In general, sequences that code for the α-amylases, starch binding domains/motifs, plastid targeting signals and other polypeptides of the invention will be at least 50%, generally at least 60%, preferably 70%, and even 80%, 85%, 90%, 95%, 98%, most preferably 99% homologous or more with the disclosed sequence. That is, the sequence similarity may range from 50% to 99% or more.

Also encompassed by the invention are fragments of the polynucleotide and polypeptide sequences of the invention. Polynucleotide fragments may encode protein fragments which retain the biological activity of the native protein. Alternatively, fragments used as hybridisation probes generally do not encode biologically active sequences. Fragments of a polynucleotide may range from at least 15, 20, 30, 50, 100, 200, 400 or 1000 contiguous nucleotides up to the full length of the native polynucleotide sequences disclosed herein.

Fragments of the polypeptides of the invention will comprise at least 5, 10, 15, 30, 50, 75, 100, 150, 200, 400 or 500 contiguous amino acids, or up to the total number of amino acids in the full length polypeptides of the invention.

Variant is also intended to allow for rearrangement, shifting or swapping of one or more nucleotides or domains/motifs (from coding, non-coding or intron regions) from genes (including α-amylases) from the same or other species, where such variants still provide a functionally equivalent protein or polypeptide of the invention or fragment thereof.

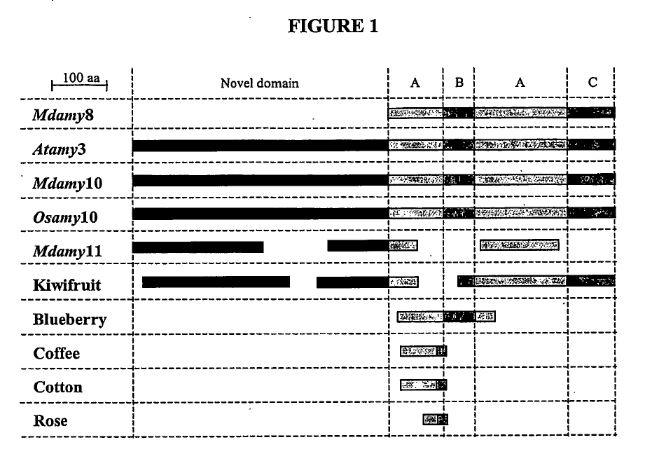

It is of course expressly contemplated that homologs to Mdamy10, Mdamy11, Atamy3, and Osamy10, exist in other plants. Such homologs are also “functionally equivalent variants” of Mdamy10, Mdamy11, Atamy3, and Osamy10, as the phrase is used herein. There are a number of examples of homologs of these genes; several are described in the experimental section. Mdamy10, Atamy3, and Osamy10 are all full-length sequences. Mdamy11 and sequences from Kiwifruit (Actizidia chinensis), Blueberry (Vaccinium corymbosum), Coffee (Coffea arabica), Cotton (Gossypium hirsutum), and Rose (Rosa woodsii), are all partial sequences. The extent of each sequence is shown graphically in FIG. 1, relative to the apple α-amylase Mdamy8, which is a cytosol-targeted α-amylase of Family two, which does not have an equivalent to the N-terminal domain of Family three α-amylases. Table 3 shows the percentage identity to Mdamy10 for each homolog, at both the nucleotide and amino acid level.

TABLE 3

| Homolog | % Nucleotide identity | % Amino acid identity |
|---|---|---|
| Mdamy11 | 91 | 86 |
| Atamy3 | 70 | 68 |
| Osamy10 | 63 | 61 |
| Kiwifruit | 70 | 71 |
| Blueberry^{a} | 81 | 80 |
| Coffee^{a} | 77 | 80 |
| Cotton^{a} | 79 | 79 |
| Rose^{a} | 81 | 81 |

^{a}These partial sequences represent the α-amylase domain of the protein, which has greater amino acid conservation than the N-terminal domain, so that they appear to produce higher amino acid identity scores, and lower nucleotide identity scores, than would be expected for their full-length sequence.

A number of ESTs representing homologs of Mdamy10, Mdamy11, Atamy3, and Osamy10, can be found in public databases. Some of these ESTs represent the N-terminal domain of Mdamy10, and include part or all of a plastid targeting signal and/or starch binding domain; examples of these ESTs (with Genbank accession numbers listed):
Arabidopsis thaliana: AV528233.1, AV530222.1, AV530101.1
Glycine max: BI698974.1, BE801868.1
Hordeum vulgare: BG299837.1
Lycopersicon hirsutum: AW616947.1, AW616948.1
Lycopersicon pennellii: AW399728.1
Medicago truncatula: BG457945.1, AW689974.1, AW690566.2, AW691339.2, BF639266.1, BF639264.1, BE322876.2, BF642292.1
Solanum tuberosum: BG598624.1
Stevia rebaudiana: BG523414.1

Other ESTs represent the α-amylase domain of Mdamy10; examples of these ESTs (with Genbank accession numbers listed):
Arabidopsis thaliana: AA042154.1, AV524237.1
Glycine max: AI938911.1, BE800170.1, BM187795.1, BM187817
Hordeum vulgare: AV933515.1, AV912828.1
Lycopersicon esculentum: AW223546.1, BE436615.1, BF176617.1, BE460819.1
Medicago truncatula: BF640476.1
Mesembryanthemum crystallinum: BE035942.1
Pinus taeda: AI725250.1
Solanum tuberosum: BE922066.1, BG598624.1
Stevia rebaudiana: BG525949.1
Zea mays: BG842601.2, BG840332.1

This EST list contains sequences from dicotyledonous and monocotyledonous angiosperms, and also from the gymnosperm Pinus taeda. It is expressly contemplated that homologs of Mdamy10, Mdamy11, Atamy3, and Osamy10, will exist, other than those listed.

A polynucleotide sequence of the invention may further comprise one or more additional sequences encoding one or more additional polypeptides, or fragments thereof, so as to encode a fusion protein. Chimeric genetic constructs including sequences encoding the novel N-terminal region of a polypeptide of the invention, or one or more starch binding domains/motifs, can be used to produce chimeric protein which can bind to starch. Systems for such recombinant expression include, but are not limited to, mammalian, bacterial, fungal and insect systems.

DNA sequences from plants other than Malus x domestica which are homologs of Mdamy10 and Mdamy11 may be identified (by computer-aided database searching) and isolated following high throughput sequencing of cDNA libraries prepared from such plants. Alternatively, oligonucleotide probes based on the sequences for Mdamy10 and Mdamy11 provided in SEQUENCE ID No's 1, 9, 11 and 13, can be synthesized and used to identify positive clones in either cDNA or genomic DNA libraries from other plants by means of hybridization or PCR techniques. Probes should be at least about 10, preferably at least about 15 and most preferably at least about 20 nucleotides in length. Hybridization and PCR techniques suitable for use with such oligonucleotide probes are well known in the art. Positive clones may be analyzed by restriction enzyme digestion, DNA sequencing or the like.

The polynucleotides of the present invention may be generated by synthetic means using techniques well known in the art. Equipment for automated synthesis of oligonucleotides is commercially available from suppliers such as Perkin Elmer/Applied Biosystems Division (Foster City, Calif.) and may be operated according to the manufacturer's instructions.

The primary importance of identification of the polypeptide/polynucleotides of the invention is that they enable modulation of starch content in plants, and particularly plant plastids such as chloroplasts. Modulation may involve a reduction in the expression and/or activity (i.e. silencing) of the polypeptide.

Any conventional technique for effecting this can be employed. Intervention can occur post-transcriptionally or pre-transcriptionally. Further, intervention can be focused upon the gene itself or on regulatory elements associated with the gene and which have an effect on expression of the encoded polypeptide. “Regulatory elements” is used here in the widest possible sense and includes other polynucleotides/polypeptides which interact with the polynucleotides/polypeptides of interest.

Pre-transcription intervention can involve mutation of the gene itself or of its regulatory elements. Such mutations can be point mutations, frameshift mutations, insertion mutations or deletion mutations. These latter mutations include so called “knock-out” mutations in which expression of the gene is entirely ablated.

Examples of post-transcription interventions include co-suppression or anti-sense strategies, a dominant negative approach, or techniques which involve ribozymes to digest, or otherwise be lethal to, RNA post-transcription of the target gene.

Co-suppression can be effected in a manner similar to that discussed, for example, by Napoli et al. (1990) and de Carvalho Niebel et al. (1995). In some cases, it can involve overexpression of the gene of interest through use of a constitutive promoter. It can also involve transformation of a plant with a non-coding region of the gene, such as an intron from the gene or 5′ or 3′ untranslated region (UTR).

Anti-sense strategies involve expression or transcription of an expression/transcription product being capable of interfering with translation of mRNA transcribed from the target gene. This will normally be through the expression/transcription product hybridising to and forming a duplex with the target mRNA.

The expression/transcription product can be a relatively small molecule and still be capable of disrupting mRNA translation. However, the same result is achieved by expressing the whole polynucleotide in an anti-sense orientation such that the RNA produced by transcription of the anti-sense oriented gene is complementary to all or part of the endogenous target mRNA.

Anti-sense strategies are described generally by Robinson-Benion et al. (1995) and Kawasaki et al. (1996).

Genetic constructs designed for gene silencing may include an inverted repeat. An ‘inverted repeat’ is a sequence that is repeated where the second half of the repeat is in the complementary strand, e.g.,

5′-GATCTA . . . TAGATC-3′
3′-CTAGAT . . . ATCTAG-5′

The transcript formed may undergo complementary base pairing to form a hairpin structure provided there is a spacer of at least 3-5 bp between the repeated regions.

Another approach is to develop a small antisense RNA targeted to the transcript equivalent to an mRNA (Llave et al., 2002) that could be used to target gene silencing.

Dominant negative approaches involve the expression of a modified plastid α-amylase polypeptide which includes a starch binding domain but lacks a catalytic domain. The result is that the protein binds starch as intended but fails to digest it, while at the same time blocking the binding of the endogenous α-amylase.

The ribozyme approach to regulation of polypeptide expression involves inserting appropriate sequences or subsequences (eg. DNA or RNA) in ribozyme genetic constructs (McIntyre 1996). Ribozymes are synthetic RNA molecules that comprise a hybridizing region complementary to two regions, each of which comprises at least 5 contiguous nucleotides of a mRNA molecule encoded by one of the inventive polynucleotides. Ribozymes possess highly specific endonuclease activity, which autocatalytically cleaves the mRNA.

Alternately, modulation may involve an increase in the expression and or activity of the polypeptide by over-expression of the polynucleotide, or by increasing the number of copies of the polynucleotide in the genome of the host.

To give effect to the above strategies, the invention also provides genetic constructs. The genetic constructs include the intended DNA (such as one or more copies of a polynucleotide sequence of the invention in a sense or anti-sense orientation or a polynucleotide encoding the appropriate ribozyme), a promoter sequence and a termination sequence (which control expression of the gene), operably linked to the DNA sequence to be transcribed. The promoter sequence is generally positioned at the 5′ end of the DNA sequence to be transcribed, and is employed to initiate transcription of the DNA sequence. Promoter sequences are generally found in the 5′ non-coding region of a gene but they may exist in introns (Luehrsen 1991) or in the coding region.

A variety of promoter sequences which may be usefully employed in the genetic constructs of the present invention are well known in the art. The promoter sequence, and also the termination sequence, may be endogenous to the target plant host or may be exogenous, provided the promoter is functional in the target host. For example, the promoter and termination sequences may be from other plant species, plant viruses, bacterial plasmids and the like. Preferably, promoter and termination sequences are those endogenously associated with the α-amylase genes.

Factors influencing the choice of promoter include the desired tissue specificity of the genetic construct, and the timing of transcription and translation. For example, constitutive promoters, such as the 35S Cauliflower Mosaic Virus (CaMV 35S) promoter, will affect the transcription in all parts of the plant. Use of a tissue specific promoter will result in production of the desired sense or antisense RNA only in the tissue of interest. With genetic constructs employing inducible promoter sequences, the rate of RNA polymerase binding and initiation can be modulated by external stimuli, such as light, heat, anaerobic stress, alteration in nutrient conditions and the like. Temporally regulated promoters can be employed to effect modulation of the rate of RNA polymerase binding and initiation at a specific time during development of a transformed cell. Preferably, the original promoters from the gene in question, or promoters from a specific tissue-targeted gene in the organism to be transformed are used. Other examples of promoters which may be usefully employed in the present invention include, mannopine synthase (mas), octopine synthase (ocs) and those reviewed by Chua et al. (1989).

The termination sequence, which is located 3′ to the DNA sequence to be transcribed, may come from the same gene as the promoter sequence or may be from a different gene. Many termination sequences known in the art may be usefully employed in the present invention, such as the 3′ end of the Agrobacterium tumefaciens nopaline synthase gene. However, preferred termination sequences are those from the original gene or from the target Malus species to be transformed.

The genetic constructs of the present invention may also contain a selection marker that is effective in cells, to allow for the detection of transformed cells containing the genetic construct. Such markers, which are well known in the art, typically confer resistance to one or more toxins. One example of such a marker is the NPTII gene whose expression results in resistance to kanamycin or hygromycin, antibiotics which are usually toxic to plant cells at a moderate concentration (Rogers et al. 1988). Alternatively, the presence of the desired genetic construct in transformed cells can be determined by means of other techniques well known in the art, such as PCR or Southern blotting.

Techniques for operatively linking the components of the inventive genetic constructs are well known in the art and include the use of synthetic linkers containing one or more restriction endonuclease sites as described, for example, by Maniatis et al. (1989). The genetic construct may be linked to a vector capable of replication in at least one system, for example, E. coli, whereby after each manipulation, the resulting genetic construct can be sequenced and the correctness of the manipulation determined.

The genetic constructs of the present invention may be used to transform a variety of plants including agricultural, ornamental and horticultural plants. In a preferred embodiment, the genetic constructs are employed to transform apple, banana, kiwifruit, pine, tomato, cotton, rose, olive, rice, blueberry, Arabidopsis, and potato plants.

As discussed above, transformation of a plant with a genetic construct including an open reading frame comprising a polynucleotide sequence of the invention wherein the open reading frame is orientated in a sense direction can, in some cases, lead to a decrease in expression of the polypeptide by co-suppression. Transformation of the plant with a genetic construct comprising an open reading frame or a non-coding (untranslated) region of a gene in an anti-sense orientation will lead to a decrease in the expression of the polypeptide in the transformed plant.

It will also be appreciated that transformation of other non-plant hosts is feasible, including well known prokaryotic and eukaryotic cells such as bacteria (e.g. E. coli, Agrobacterium), fungi, insect, and animal cells is anticipated. This would enable production of recombinant polypeptides of the invention or variants thereof. The use of cell-free systems (e.g. Roche Rapid Translation System) for production of recombinant proteins is also anticipated (Zubay, 1973).

The polypeptides and proteins of the invention produced in any such hosts may be isolated and purified from same using well known techniques.

Techniques for stably incorporating genetic constructs into the genome of target plants are well known in the art and include Agrobacterium tumefaciens mediated introduction, electroporation, protoplast fusion, injection into reproductive organs, injection into immature embryos, high velocity projectile introduction, floral dipping and the like. The choice of technique will depend upon the target plant to be transformed.

Once the cells are transformed, cells having the genetic construct incorporated into their genome may be selected by means of a marker, such as the kanamycin resistance marker discussed above. Transgenic cells may then be cultured in an appropriate medium to regenerate whole plants, using techniques well known in the art. In the case of protoplasts, the cell wall is allowed to reform under appropriate osmotic conditions. In the case of seeds or embryos, an appropriate germination or callus initiation medium is employed. For explants, an appropriate regeneration medium is used.

In addition to methods described above, several methods are known in the art for transferring genetic constructs into a wide variety of plant species, including gymnosperms angiosperms, monocots and dicots (see, e.g., Glick and Thompson (1993) Birch (1997) and Forester et al. (1997)). For a review of regeneration of trees, see Dunstan et al. (1995).

The resulting transformed plants may be reproduced sexually or asexually, using methods well known in the art, to give successive generations of transgenic plants.

The nucleotide sequence information provided herein will also be useful in programs for identifying nucleic acid variants from, for example, other organisms or tissues, particularly plants, and for pre-selecting plants with mutations in Mdamy10 or Mdamy11 or their equivalents which renders those plants useful in an accelerated breeding program to produce plants in which starch content is modulated. More particularly, the nucleotide sequence information provided herein may be used to design probes and primers for probing or amplification of Mdamy10, Mdamy11 or variants thereof. An oligonucleotide for use in probing or PCR may be about 30 or fewer nucleotides in length. Generally, specific primers are upwards of 14 nucleotides in length. For optimum specificity and cost effectiveness, primers of 16-24 nucleotides in length are preferred. Those skilled in the art are well versed in the design of primers for use in processes such as PCR.

If required, probing can be done with entire restriction fragments of the gene disclosed herein. Naturally, sequences based upon FIG. 2 or the complements thereof can be used.

Such probes and primers also form aspects of the present invention.

Methods to find variants of the of polynucleotides of the invention from any species, using the sequence information provided by the invention, include but are not limited to, screening of cDNA libraries, RT-PCR, screening of genomic libraries and computer aided searching of EST, cDNA and genomic databases. Such methods are well known to those skilled in the art.

The invention will now be illustrated with reference to the following non-limiting experiments.

### EXPERIMENTAL

Materials and Methods

Oligonucleotides used in this work:

Sequences are from 5′ to 3′ end:

| | |
|---|---|
| SP1 | AACGGTGATTCAGAACAGCATC |
| SP2 | ATGTACCCTTGAGGCGACACAG |
| SP3 | GGTGGGAACCAAATCACAGTG |
| Oligo-dT | GACTCGAGTCGACATCGATTTTTTTTTTTTTTTTT |
| Race11 | GACTCGAGTCGACATCG |
| Atamy1exon3F | GTTACTACCGGGAAAGCTATAC |
| Atamy1exon3R | GGGCGCTCCATCAAAATC |
| Atamy2exon5F | TCTTCCACAGGACCTTTATTC |
| Atamy2exon5R | AGTCCTCCGGTACAAGAAGTC |
| Atamy3exon8F | GGAACAATTGATGAGCTAAAAGATAC |
| Atamy3exon8R | GGAAATGAGGGTCATCTGC |
| Amyten12-Xh | TCTCGAGCGTACCATGTCGACGGTTAG |
| Amyten13r-Kp | AAGGTACCCATCTTTCCCTCCACCACTTC |
| Amyten14r-Kp | AAGGTACCTCTGTCACGTAGCTGAGCATC |
| NewUNIf2 | ATATTRTGCCAAGGTTTTAACTGGGA |
| NewUNIr2 | WARRCGWCCWCCAAAWAKATTCCA |

Sampling of Plant Material

Mature apple fruit (Malus x domestica) were harvested from mature apple trees in Auckland, New Zealand. These fruit were placed in standard commercial boxes and stored at 4° C. for eight days. Tissue samples were taken and frozen immediately in liquid nitrogen and stored at −80° C. until being used for extraction of RNA.

RNA Extractions

Between 2 g (leaf, flower, young fruit) and 10 g (mature fruit) of tissue were ground under liquid nitrogen, added to 15 mL of heated lysis buffer, and RNA extracted according to the method of Langenkamper et al. (1998). cDNA was prepared from mRNA in a standard manner.

EST Genetic Construction and Sequencing

cDNAs were cloned into standard library vectors eg Lambda Zap 2 and Lambda Zap Express (Stratagene). Individual clones were excised and plasmid DNA prepared using standard methods. Sequence was then determined by sequencing from both the 5′ and 3′ ends in the standard manner and intermediate sequences were determined using PCR primers designed to the known sequence in a standard manner.

5′ RACE (Rapid Amplification of cDNA Ends)

5′ RACE was performed on RNA from apple floral buds, using three Mdamy10 specific primers, SP1, SP2, and SP3, located at what was the 5′ end of the Mdamy10 sequence at the time. The three primers are reverse-complements of the Mdamy10 coding sequence, and are nested; SP1 is the most 3′ sequence, SP3 the most 5′.

SP1 was used to perform the initial reverse transcription step, performed by Superscript II Reverse Transcriptase (Invitrogen) at 42° C. for 30 min. The product was digested with RNase H and then purified with a PCR purification kit (Qiagen), before having a poly-A tail added with terminal transferase and dATP. The first PCR round used the SP2 and RACE oligo-dT primers, and was performed with Expand Hi-Fidelity enzyme (Roche). The resulting mix was again cleaned up with the Qiagen PCR purification kit and then used as template for the final PCR round, with SP3 and RACE1 primers. The products of 5′ RACE were cloned into pGEM-T Easy (Promega) and sent for sequencing.

RT-PCR of Arabidopsis α-Amylase Genes:

Arabidopsis RNA samples were treated with DNase I (Roche) and then amplified using the Titan One Tube RT-PCR System (Roche), using the primer pairs Atamy1exon3(F/R), Atamy2exon5(F/R), and Atamy3exon8(F/R). These primer pairs amplify the Arabidopsis genes Atamy1, Atamy2, and Atamy3, respectively, within domain 13. The reaction mix was incubated at 42° C. for 30 min, before undergoing 30 rounds of standard PCR cycling conditions.

Amplification of Family Three Gene Sequences from Other Plant Species

Genomic DNA was extracted from a number of plant tissues, including: needles of pine tree (Pinus radiata); sprouting potato tubers (Solanum tuberosum); sprouting onion bulbs (Allium cepa); rose petals (Rosa woodsii); olive leaves (Olea europaea); coffee leaves (Coffea arabica); banana leaves (Musa acuminata); and mango fruit (Mangifera indica). Extraction was performed with a Dneasy Plant Mini Kit (Qiagen), according to the manufacturers instructiosn. DNA from cotton (Gossypium hirsutum) was kindly supplied by Dr John Lunn (CSIRO Plant Industry, Canberra, Australia).

PCR was performed with Expand Hi-Fidelity enzyme (Roche), using primers NewUNIf2 and NewUNIr2. These degenerate primers were designed from an alignment of Atamy3, Mdamy10, and Osamy10, to amplify only Family three genes, but from a wide range of species. The products of PCR were cloned into pGEM®-T Easy (Promega) and sequenced.

Computational Analysis of Sequence Information:

Computational analysis was performed using the Wisconsin Package Version 10.1, (Genetics Computer Group (GCG), Madison, Wis.). Sequence identity was calculated using the pairwise alignment program Gap, which uses the algorithm of Needleman and Wunsch (J. Mol. Biol. 48; 443-453 (1970)). The default parameters were used: Gap creation penalty: 50 (for nucleotide input); or 8 (for amino acid input); Gap extension penalty: 3 (nucleotide); 2 (amino acid). Amino acid sequence alignments were performed using the program CLUSTALW (Thompson et al., 1994), and trimmed and shaded using the program GeneDoc (Nicholas and Nicholas, 1997).

Prediction of Subcellular Localisation of Family Three α-Amylases

The subcellular localisations of Mdamy10, Mdamy11, Atamy3, and Osamy10, were predicted using servers at the Center for Biological Sequence Analysis (http://www.cbs.dtu.dk/services/). The program Target P (Emanuelsson et al., 2000) was used initially to predict the targeting of the proteins. The program ChloroP (Emanuelsson et al. 1999) was then used to predict the length of plastid targeting signal for each protein.

Genetic Construction of Plasmids for Expression in Plants and Escherichia coli

Mdamy10 sequences were cloned or amplified from HortResearch EST genetic construct ‘62629’. GFP fusion genetic constructs were produced by cloning PCR products into the plasmid pDS-GFP-ART7, which has the gene encoding smGFP (Davis and Vierstra, 1998), driven by the CaMV 35S promoter. The smGFP gene was cloned into pDS-GFP-ART7 so as to leave a XhoI and a KpnI restriction endonuclease site upstream of the smGFP gene, with the KpnI site in-frame with the ATG start of the gene. E. coli expression vectors were produced in the vectors pET-30a and pET-30b (Novagen).

PCR was used to amplify the fragments ‘cTP’ and ‘SBD’ for fusion to smGFP. The cTP (plastid targeting peptide) product was amplified using primers Amyten12-Xh, located at the 5′ end of the coding sequence, containing a XhoI restriction endonuclease site upstream of the ATG start of Mdamy10, to facilitate cloning; and Amyten13r-Kp, located approximately 300 bp downstream of the ATG start site, containing a KpnI restriction endonuclease site, in frame with open-reading frame of Mdamy10. This amplified region encodes the plastid targeting signal of Mdamy10, plus another 35 amino acids following the signal.

The ‘SBD’ (starch binding domain) product was amplified using primers Amyten12-Xh, the same primer as for the cTP product; and Amyten14r-Kp, located approximately 1250 bp downstream of the ATG start site, containing a KpnI restriction endonuclease site, in frame with open-reading frame of Mdamy10; this primer also removes a naturally occuring KpnI site found in the Mdamy10 sequence. This amplified region encodes the putative cTP, plus both copies of the putative starch binding domain motif pair.

Following PCR, both products were cloned into the vector pGEM®-T Easy (Promega), and excised by digestion with XhoI (New England Biolabs) and KpnI (Roche) restriction endonucleases. The restriction products were then cloned into pDS-GFP-ART7, which had been similarly digested with XhoI and KpnI. The resulting plasmids were termed pcTP-ART7 and pSBD-ART7. The expression regions of these two genetic constructs were excised by digestion with NotI and subsequent cloning into the vector pART27, which had also been cut with NotI. This produced the final expression vectors, pcTP-ART27 and pSBD-ART27.

Mdamy10 was removed from EST genetic construct 62629 by digestion with restriction endonucleases EcoRI and XhoI. This removed the first 220 bp of the Mdamy10 coding sequence, including the cTP encoding region of the cDNA and a further 13 amino acids. This fragment was cloned into pET-30a, which had been cut using the same enzymes, creating the genetic construct pAmy10ET-30a. This added a short peptide to the N-terminus of Mdamy10, which includes a His₆ tag, allowing for nickel ion affinity purification.

SmGFP was cloned from pDS-GFP-ART7 into pET-30b using KpnI and HindIII restriction endonuclease sites. An Mdamy8-GFP fusion (Nterm-GFP) was also cloned into pET-30b in this manner. SBD-GFP was cloned into pET-30a from pSBD-ART27 using EcoRI and HindIII restriction endonuclease sites, thereby removing the cTP domain from the genetic construct. Again, all cloning strategies added the His₆ tag to the N-terminus of the expressed proteins.

Preparation of Competent Agrobacterium tumefaciens GV3101:

A single colony of Agrobacterium, which had been grown on an LB plate containing 25 μg/mL of gentamycin, was used to inoculate 10 mL of liquid LB media, also containing 25 μg/mL of gentamycin. The culture was incubated for 48 hr with shaking at 200 rpm in a 28° C. incubator. The cells were harvested by centrifugation (3800×g for 5 min at 4° C.) and resuspended in 50 mL of ice-cold 10% glycerol. The cells were washed twice more in glycerol, before finally being resuspended in 1 mL of ice-cold 10% glycerol and aliquoted into 45 μL in microcentrifuge tubes. The aliquots were stored at −80° C.

Transformation of Agrobacterium tumefaciens GV3101

45 μL aliquots of competent Agrobacterium cells were thawed gently on ice. 50-200 ng of plasmid DNA was added to each aliquot and gently mixed, then 40 μL of the cell/plasmid mixture was pipetted into a pre-chilled electroporation cuvette (0.2 cm gap, Bio-Rad). The cells were electroporated using a BioRad GenePulser, on the following settings:
Voltage: 2.5 kV
Capacitance: 25 μFd
Resistance: 400 Ohms

The time constant for the pulse was typically 7-9 ms.

The cells were immediately recovered by addition of 1 mL LB media, then decanted into sterile 15 mL centrifuge tubes and incubated at room temperature, with shaking (60 rpm). After 2 hours, 10 μL and 100 μL of the transformed bacteria was spread onto separate LB plates containing rifampicin (10 μg/mL); gentamycin (25 μg/mL); and spectinomycin (100 μg/mL); then grown for 48 hours at 28-30° C.

Transient Transformation of Nicotiana benthamiana Leaves

Transformation was performed by the method of Hawes et al. (2000). Agrobacterium tumefaciens were grown in 5 mL cultures of LB media containing rifampicin (10 μg/mL); gentamycin (25 μg/mL); and spectinomycin (100 μg/mL) for 48 hours. The cells were collected by centrifugation and resuspended, to a final OD₆₀₀ of 0.5-0.6, in infiltration medium: 50 mM MES pH 5.6, 0.5% (w/v) glucose, 2 mM Na₃PO₄, 100 μM acetosyringone (added fresh from 200 mM stock in DMSO). The bacterial suspension was injected through the stomata on the underside of N. benthamiana leaves, using a “blunt” 1 mL syringe (i.e. with no needle attached). The leaves were detached and kept in dark, moist conditions at room temperature for 2-3 days.

Microscopy

Leaf samples were examined using an Olympus Vanox AHBT3 microscope. Fluorescence was detected using one of two filter sets:
UV filter set—Olympus code BH2-DMU: excitation 320-380 nm, emission 420 nm.
GFP plant—Omega filter set XF100-2: excitation 455-495 nm, emission 515-560 nm.
   Expression of Protein in E. coli

All pET-30 based expression vectors were transformed following the manufacturer's instructions into BL21 CodonPlus RIL cells (Stratagene) for expression. A single colony was picked from a plate and used to inoculate 15 mL of 2YT media (16 g/L bactotryptone, 10 g/L yeast extract, 5 g/L NaCl, pH 7), containing 30 μg/ml kanamycin and 50 μg/mL chloramphenicol. Cultures were grown overnight at 37° C., with vigourous shaking (150 rpm). Each 5 mL of the 15 mL culture was used to seed one of three 330 mL aliquots of 2YT (including 30 g/mL kanamycin and 50 μg/mL chloramphenicol), in 1 L flasks. These were shaken at 37° C. for 3-4 hours, until the OD₆₀₀ of the cultures had reached ˜1.0, then the flasks and incubator were cooled to 18° C. and expression was induced by addition of IPTG to a final concentration of 0.5 mM. Cells were grown at 18° C. with continual shaking, for a further 24 hours before harvesting.

Extraction of Soluble Protein from E. coli Cells.

Cells were harvested by centrifugation (2500×g for 10 min); cells from 1 L of culture were then resuspended in 20 mL of the appropriate buffer. For α-amylase activity gels, the cells were resuspended in activity buffer (100 mM Hepes pH 7.5, 5 mM calcium acetate, 10 mM DTT); GFP genetic constructs were resuspended in binding buffer (5 mM imidazole, 0.5 M NaCl, 20 mM Tris-HCl pH 7.9). All buffers contained one Complete protease inhibitor tablet, EDTA free (Roche) per 50 mL of buffer.

The bacterial cells were lysed by twice passing the 20 mL of cell suspension through a French Pressure Cell Press (American Instrument Co. Inc, Silver Spring, Md., USA), at pressure of 12,700 psi. The lysate was centrifuged at 12000×g for 20 min, and then passed through a filter 0.45 μm pore size. The resulting supernatant was termed the crude extract.

Partial Purification of Recombinant Protein from Crude Extract

For the protein extractions performed in α-amylase buffer, approximately 10 mL of crude extract was applied to a PD-10 gel filtration column that had been pre-equilibrated with binding buffer. GFP samples were not subject to buffer exchange. Purification was performed on a 5 mL Hi-Trap Chelating HP column (Amersham-Pharmacia Biotech) that had been charged with NiSO₄. 10 mL of the extract was applied to the column, and washed according to the manufacturer's instructions. Recombinant protein was eluted into 2.5 mL fractions with elution buffer (1 M imidazole, 0.5 M NaCl, 20 mM Tris-HCl pH 7.9). α-Amylase and pET-30a were immediately exchanged into activity buffer using a PD-10 column.

Denaturing Gel Electrophoresis and Western Analysis of Protein Samples

Crude extract and certain fractions from the protein purification steps, were analysed by standard procedures on a SDS-PAGE gel, consisting of a 10% polyacrylamide separating gel and a 4% polyacrylamide stacking gel. Following electrophoiesis, gels were either stained with colloidal coomassie protein stain or transferred to Immobilon-P PVDF membrane (Millipore) by semi-dry electophoresis. For detection of the His₆ motif, the blots were incubated with anti-His₆ monoclonal antibody (Roche), followed by anti-Mouse IgG-alkaline phosphatase (Stressgen) secondary antibodies. For detection of the GFP motif, blots were incubated with anti-GFP polyclonal antibody, IgG fraction (Molecular Probes), followed by anti-Rabbit IgG-secondary antibodies. Both types of blot were detected using 1-STEP™ NBT/BCIP alkaline phosphatase detection reagent (Bio-Lab laboratories).

Protein Extraction from Apple and Arabidopsis

Leaf samples were removed from seedling apples and full rosette plants of Arabidopsis at the end of a 12 hour light cycle. Leaves were frozen in liquid nitrogen and ground using a mortar and pestle, then were added to extraction buffer (50 mM Hepes pH 7.8, 5 mM calcium acetate, 5 mM magnesium chloride, 10 mM DTT, 0.5% (w/v) PVPP). The samples were then filtered through 2 layers of Miracloth (Calbiochem, La Jolla, Calif., USA) and centrifuged at 14,000×g for 10 mins. The supernatant was immediately loaded onto native PAGE gels.

Native Gel Electrophoresis

Crude extract and certain fractions from the protein purification steps, were analysed on two types of native PAGE gels. Starch gels that contained 2% amylopectin in the separating gel. Samples were electrophoresed for 16-20 hrs at a constant current of 10 mA, and following electrophoresis, the gels were washed in distilled water and incubated for 24 hrs in activity buffer 2 (50 mM Hepes pH 7.8, 5 mM calcium acetate, 5 mM magnesium chloride, 10 mM DTT, 20 (g/mL cycloheximide). Starch hydrolysis was detected by staining with iodine solution (180 mM potassium iodide, 50 mM iodine), followed by destaining with fixative (30% methanol, 5% acetic acid). Starch gels were stored at 4° C., covered by Saran plastic wrap.

Samples were also run on native PAGE gels without amylopectin, for 3-4 hours at a constant current of 20 mA. Protein was then transferred into a separating gel containing 2% amylopectin, by capillary blotting with activity buffer 2. After overnight transfer, the amylopectin containing gel (termed the starch transfer gel) was washed and incubated for a further 24 hours in activity buffer 2. Staining, de-staining and storage was the same as for starch gels.

Results

Identification of the Mdamy10 gene from Apple:

To identify α-amylase genes from Apple, an EST database, containing sequences derived from a number of different apple tissues, was compared to the coding sequence of the known apple α-amylase gene, Mdamy8 (Wegrzyn et al., 2000). The vast majority of α-amylase-like sequences discovered within the HortResearch database corresponded to a previously uncharacterised α-amylase gene (first identified 17 Jul. 2000). Transcripts of this gene are present in numerous tissue libraries, including floral bud, petal, young fruit and mature fruit libraries. Blast searches against the Genbank database revealed a match to a putative α-amylase from Arabidopsis, as well as to a number of ESTs from a variety of plants, including Arabidopsis, Lycopersicon esculentum (tomato), Medicago truncatula, Glycine max (soybean), Solanum tuberosum (potato) and Pinus taeda (loblolly pine). The full-length cDNA sequence of this α-amylase, named Mdamy10 by the applicants, was obtained by sequencing of a single, full-length EST clone, EST 62629, along with the identification of overlapping sequences from partial ESTs. The coding region of Mdamy10 is 2706 bp long, encoding 901 amino acids. The last 400 amino acids of Mdamy10 show modest identity to both Mdamy8 (46%) and an α-amylase from Vigna mungo, any Vm1 (48%); however the first 500 amino acids are not found in any characterised α-amylase (FIG. 1).

Identification of a Homologue of Mdamy10 in Apple:

The only apple EST sequence that matched the 5′ region of Mdamy10 was from a full length cDNA, displaying 79% nucleotide identity, but only 900 nucleotides in length (FIG. 1). The transcript appears to be the product of a gene homologous to Mdamy10, except that the equivalent of the third intron of Mdamy10 (Table 1) has not been spliced from the transcript. This results in cleavage and polyadenylation of the transcript within the third intron, and potential production of a truncated protein. We have tentatively labelled the transcript Mdamy11.

We have two further fragments of Mdamy11 sequence. The first was produced with a primer pair designed to Mdamy10 cDNA sequence, which amplified two products of slightly different sizes (1028 bp and 831 bp) from genomic DNA. The fragments had very similar exon sequences (95% identity at both nucleotide and amino acid levels), and each contained three introns, found in the same positions. However the introns of the two fragments differ significantly in their sequence (80% identity at nucleotide level) and their size (602 bp total, versus 405 bp). The larger fragment (and hence larger intron) is from Mdamy10, the smaller is presumed to be from Mdamy11. The sequence is from close to the 3′ end of the α-amylase domain, and so does not overlap the Mdamy11 transcript (FIG. 1).

A final additional fragment was isolated during 5′ RACE (rapid amplification of cDNA ends) of Mdamy10 from apple floral bud RNA. The 600 bp region overlapped with sequence from Mdamy10, with 6 bp in disagreement within the 95 bp of overlap. These were initially thought to be sequence errors, accumulated from both EST genetic construction and 5′ RACE, however attempts to continue upstream of this region using 5′ RACE were unsuccessful. Finally, sequence from the full-length EST 62629 reached the region of the 5′ RACE fragment, and was found to share only 86% identity at the nucleotide level. The 5′ RACE fragment was automatically removed from the Mdamy10 sequence contig. This fragment does not overlap either of the previous Mdamy11 sequences, but corresponds to the junction of the novel N-terminal domain and the α-amylase domain of Mdamy10 (FIG. 1).

These three fragments of Mdamy11, when taken together, cover 61% of the total coding sequence of Mdamy10, with 86% amino acid identity between the two genes through these regions. The identity is much greater in the C-terminal (α-amylase) domain (96%) than in the N-terminal domain (79%).

Identification of a Homologue of Mdamy10 in Arabidopsis:

Atamy3 was originally annotated in Genbank by TIGR, predicted from genomic sequence. The annotation suggested a coding sequence 2478 bp in length, encoding a protein of 826 amino acids (compared to around 415 aa for other plant α-amylases). We reanalysed the appropriate chromosomal segment, using Eukaryotic GeneMark.hmm, (LUKASHIN & BORODOVSKY, 1998) to predict gene splicing. This algorithm predicted an even larger coding region of 2661 bp (FIG. 4), due to inclusion of two extra exons, producing a protein 887 aa in size (FIG. 5), displaying 68% identity to the predicted Mdamy10 product (FIG. 1). The N-terminal domain (bold and underlined in FIG. 5) has 56% identity to Mdamy10, while the rest of the protein has 82% identity to Mdamy10. The polynucleotide sequences display 70% identity.

Our sequence prediction was confirmed by the sequencing of an Atamy3 cDNA clone at the Salk Institute, Calif., published in Genbank on 21 Aug. 2001 (accession AY050398). Atamy3 is located on chromosome 1 (BAC T17F3). Blast searches have not yielded any other Family three α-amylase genes in the Arabidopsis genome.

Identification of a Homologue of Mdamy10 in Rice (Oryza sativa):

Osamy10 was originally discovered by using the BlastP program to search the genbank non-redundant database, using Mdamy10 as the query sequence. The sequence was annotated in Genbank by members of the rice genome research program as a ‘putative alpha-amylase’; the coding sequence had been predicted from genomic sequence (accession AP003408-23 May 2002). As with Atamy3, we reanalysed the appropriate chromosomal segment, using Eukaryotic GeneMark.hmm, to predict gene splicing. This revealed misannotation of the gene, namely the inclusion of a 99 bp DNA segment which was in fact part of an intron (this was confirmed by comparing the relative intron positions of Atamy3). The sequence predicted by Eukaryotic GeneMark.hmm is 2619 bp (FIG. 6), encoding 873 amino acids (FIGS. 1 and 7). The polynucleotide sequence displays 63% identity to Mdamy10. The protein sequence displays 61% identity to Mdamy10, over its full length, however the N-terminal domain (bold and underlined in FIG. 7) has only 44% identity to Mdamy10, while the rest of the protein has 80% identity to Mdamy10.

Genomic Sequences from Other Plant Species:

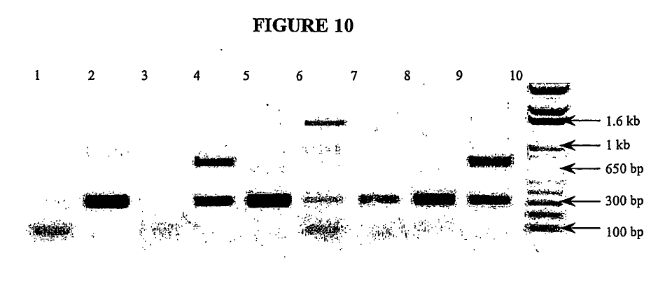

PCR was performed with degenerate primers, to amplify Family three sequences from the genomic DNA of a number of plant species. FIG. 10 shows the results of PCR. All DNA samples except onion produced a molecule of about 320 bp, which was the size of product expected from amplification of Mdamy10 cDNA with the primer pair. Cloning and sequencing of several of these products confirmed them to be Mdamy10 cDNA sequence, presumably caused by aerosol contamination. The abundance of this product inhibited cloning of other PCR products in the samples, but subsequent experiments failed to pinpoint and/or remove the source of contamination.

Most DNA samples produced at least one other DNA molecule larger than the 320 bp band. At the time of writing, only three of these products have been successfully cloned; an approximately 800 bp product from rose (only partially sequenced) (FIG. 10., lane 4), a 962 bp product from coffee (FIG. 10., lane 6), and an 808 bp product from cotton (FIG. 10., lane 9). All three sequences contain a single intron, which accounts for almost all of the size difference between the products of different species. The position of each intron is conserved between the three products, and correspond to the seventh intron of Atamy3 (intron 9 of Table 1). This intron position is not found in α-amylases from Families one or two. There were no introns found in conserved positions of Family one and Family two introns.

The three fragments all cover a region of the α-amylase genes that encodes part of the previously characterised domains A and B of α-amylases (FIG. 1). The introns were removed from the sequences and the resulting segments were translated into the expected polypeptides. Both the polynucleotide and polypeptide sequences were aligned with representatives of each of the three α-amylase families; Mdamy10 (Family three), Mdamy8 (Family two), and AmyVm1 (Family one). The degree of identity between each pair was calculated as a percentage (Table 4).

TABLE 4

| | | |
|---|---|---|
| Mdamy10 | Mdamy8 | AmyVm1 |

| % Identity | DNA | AA | DNA | AA | DNA | AA |
|---|---|---|---|---|---|---|
| Coffee | 77 | 80 | 51 | 42 | 56 | 56 |
| Cotton | 79 | 79 | 51 | 40 | 52 | 48 |
| Rose | 81 | 81 | 47 | 21 | 54 | 44 |

All three fragments show significantly higher identity towards Mdamy10 than they do towards α-amylases from other families; particularly at the amino acid level.

Structure of α-Amylase and α-Amylase-Like Genes in Plants:

The gene structures of plant α-amylases have been compared previously, most significantly in HUANG et al. (1990). Repeating this type of analysis, but using new Arabidopsis, apple and rice α-amylase sequences, produces a far more complicated picture of α-amylase intron evolution. Previously sequenced α-amylase genes have at most three introns. Some cereal genes appear to have lost the second intron (HUANG et al., 1990), but the position of each intron is conserved between genes and species.

The intron/exon structure of Mdamy10 is significantly different from previously sequenced α-amylase genes (Table 1). The gene contains 12 introns, compared to other characterised plant α-amylases, which have 2 or 4 introns. Of the 12 introns that interrupt the coding sequence of Atamy3, 6 of them are within the α-amylase-encoding region of the gene (i.e. the 3′ half). None of the introns of the Family three α-amylases correspond to introns of the Family one α-amylases.

Post-Transcriptional Processing of α-Amylase-Like Genes:

In contrast to the Mdamy8 transcripts, Mdamy10, Mdamy11 and Atamy3 all appear to have short 5′ UTRs—between 19 bp and 46 bp. However, Mdamy10 has a very long 3′ UTR, up to 557 bp, although one of the four different polyadenylation sites can produce a 3′ UTR as short as 428 bp. The 3′ UTR sequence of Atamy3 is only 220 bp.

Subcellular Localisation of Plant α-Amylases:

All of the Family three proteins were identified as plastid-targeted; the program ChloroP (EMANUELSSON et al., 1999) predicted transit peptide lengths of 61 amino acids (Mdamy10), 70 amino acids (Mdamy11), 55 amino acids (Atamy3), and 53 amino acids (Osamy10). To test this, the plastid targeting sequence of Mdamy10 was fused to green fluorescent protein and transiently expressed in the leaves of N. benthamiana. The pattern of GFP localisation was compared to GFP alone.

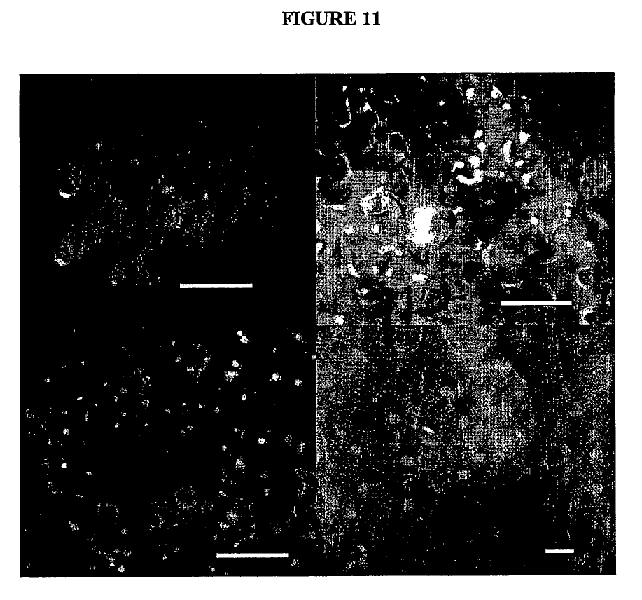

GFP is localised to the cytosol of epidermal cells (FIG. 11), and also to the nucleus (visible in the very centre of epidermal cells, top left panel, FIG. 11), but not to the vacuole, which dominates the cell's volume. By comparison, the cTP-GFP fusion localises to multiple organelles, discoid in shape, within each cell (FIG. 11, top right and lower panels). Each disc has a maximum diameter of about 41 μm, as compared to the nucleus, which is about 10 μm in diameter. The same organelles display chlorophyll autofluorescence (red) under UV illumination (FIG. 11, top right panel), identifying them as chloroplasts; co-localisation of the GFP and chlorophyll appears as an orange/yellow (only under UV illumination).

A second fusion genetic construct, SBD-GFP, was produced, containing the majority of the N-terminal domain (including the cTP) of Mdamy10, also fused to GFP. This too was expressed in N. benthamiana leaves, but produced no detectable fluorescence. It was unclear whether this was due to improper folding of the GFP domain or because of degradation of the protein in plant cells. We attempted to determine localisation of the fusion protein by immunological detection using anti-GFP antibodies, but could not detect any significant signal from GFP.

Expression of GFP Fusion Proteins in E. coli:

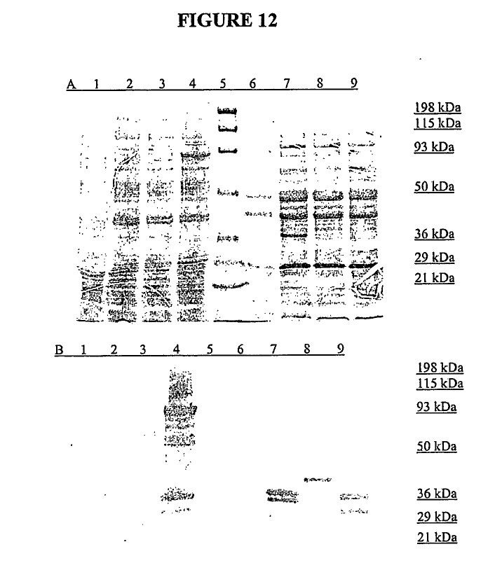

The inserted sequences of pDS-GFP-ART7, pDS-Nterm-ART7 (GFP fused to the N-terminus of Mdamy8), and pSBD-ART7 were transferred into pET-30 vectors and expressed in E. coli. The cTP of Mdamy10 was removed from the SBD fusion genetic construct, at a naturally occurring EcoRI site, so as to increase the overall solubility of the protein. After induction with IPTG, samples of the cultures were examined by fluorescence microscopy; green fluorescence was observed for all three proteins, although pGFP-ET-30b produced more intense fluorescence than the other two samples; pET-30a alone produced no fluorescence. This was also true of the soluble protein fractions extracted from the cultures. Extracts were made of the soluble and insoluble fractions of the cell, and the samples were subjected to SDS-PAGE and western analysis as shown in FIG. 12.

These analyses show that pGFP-ET-30b produced the most soluble, full-length (31 kDa) protein; this corresponded to the highest level of fluorescence. pNterm-ET-30b produced almost no soluble, full length protein (78 kDa), whilst pSBD-ET-30a had a small amount of soluble full length product (71 kDa). All fusion proteins appear slightly larger on the gel than their expected sizes. The vast majority of protein produced by pSBD-ET-30a was found in the insoluble fraction. Both of the fusion genetic constructs produced smaller, soluble products, which may be breakdown products of GFP or may come from internal initiation with the fusion gene, leading to translation of an unfused GFP molecule. This GFP may be responsible for the fluorescence seen in induced cultures.

Expression of α-Amylase Mdamy10 in E. coli:

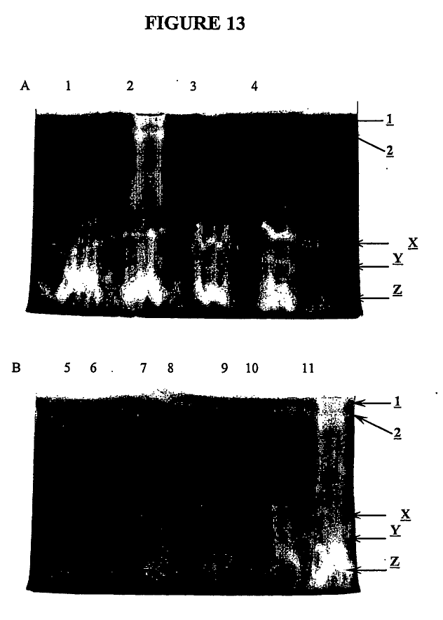

Mdamy10 protein was expressed in E. coli at low temperatures, and the protein was extracted and partially purified on a nickel column. Both purified and unpurified protein were electrophoresed into polyacrylamide gels containing 2% amylopectin; these gels sort proteins based on their affinity for the amylopectin, proteins with low affinity travel further than high affinity proteins. The gels were stained with iodine, which forms a purple complex with amylopectin; a clear area on the gel indicates hydrolysis of the substrate (FIG. 13).

The gels revealed a number of intrinsic E. coli proteins with hydrolytic activity, which can be seen in protein extracts from pET-30a expressing bacteria (labelled X, Y and Z in FIG. 13). The same intrinsic activity was visible in bacterial cultures expressing pAmy10-ET-30a, but there were also two extra 2 bands close to the boundary of the stacking and separating gels (labelled 1 and 2 in FIG. 13). These bands were visible only in crude protein extracts; desalting or purifying on a nickel column led to loss of this activity.

Crude extract of Mdamy10 protein was examined on a starch transfer gel, alongside whole protein samples from apple and Arabidopsis leaves. In this analysis, proteins are sorted based upon size and charge, rather than their affinity for starch.

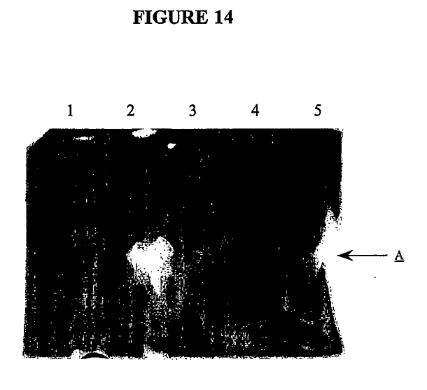

FIG. 14 shows a starch transfer gel that has been stained with iodine. Extracts from E. coli containing pAmy10-ET-30a show a band of amylolytic in the middle of the gel (labelled A in FIG. 14, lanes 2 & 5) that is not present in the pET-30a only control. A faint band of the same size can be seen in extracts of apple leaf tissue (lane 3, FIG. 14). The intrinsic E. coli activity seen in starch gels is visible only at the very bottom of the starch transfer gels, presumably they have high mobility in the native PAGE gels and have mostly eluted from the gel prior to transfer.

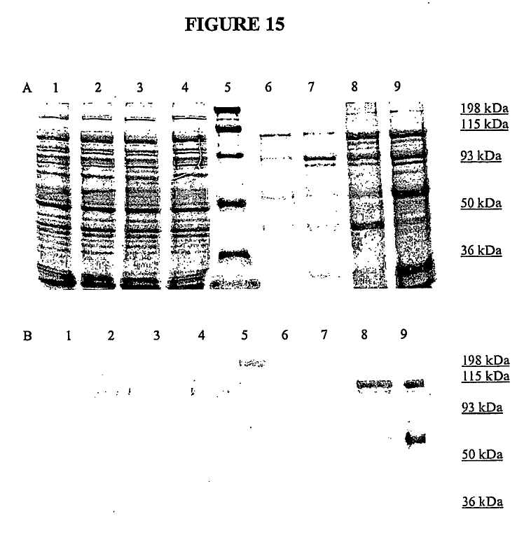

We undertook SDS-PAGE of the various protein fractions, followed by western blotting using anti-His₆ antibodies (FIG. 15). The expected size of the Mdamy10 protein plus the 6×His tag of pET-30a is 99 kDa. A 105 kDa protein, with a 6×His tag was expressed only in pAmy10-ET-30a cultures, and was successfully recovered by nickel column purification. The difference between the size of expected and expressed protein is probably due to the inherent inaccuracy of determining size in this manner. There were also a few secondary products purified, the major protein was around 55 kDa in size. This fragment eluted mostly in the third 2.5 mL elution fraction, while the 105 kDa protein eluted in the second and third fractions. The 55 kDa product is probably responsible for the lower band (band 2) in the Mdamy10 lane of starch gels (FIG. 13), as the intensity of band 2 appears to be relative to the amount of 55 kDa protein in the extract (data not shown). It appears Mdamy10 was inactivated during purification, rather than being degraded or misplaced.

Expression Profile of Mdamy10 and Homologues in Plants:

ESTs encoding Mdamy10 have been sequenced from a wide variety of apple tissues, including:
Apple fruit skin peel, from tree-ripened fruit 150 days after full bloom (DAFB).
Apple fruit cortex tissue, from tree-ripened fruit 150 DAFB.
Young apple fruit, 10 DAFB.
Young apple fruit, 24 DAFB.
Apple fruit stored at 0.5° C. for 24 hours.
Spur buds from apple trees.
Apple pre-opened floral bud.
Apple leaf infected with Venturia inaequalis.

ESTs from kiwifruit were found in several tissues from ripe fruit, including skin and inner cortex, and also in breaking bud. A single EST was isolated from the skin of blueberry (Vaccinium corymbosum).

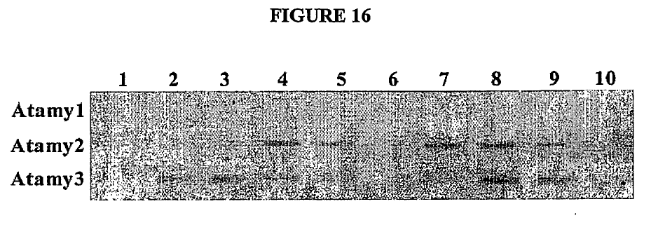

Some RT-PCR experiments were performed on a developmental series of tissues from Arabidopsis. The experiment used primers specific to each of the three Arabidopsis genes to ascertain the relative expression of each family (FIG. 16).

The RT-PCR shows that Atamy1 has very low expression levels in Arabidopsis (transcript was detectable in some tissues after 40 rounds of PCR (data not shown)), whilst Atamy2 and Atamy3 have similar levels of expression, but different patterns of expression. Atamy3 expression was at its highest in the leaves of growing stems, and moderate in a number of other tissues (emerging cotyledons, whole seedlings, and young seed pods).

Discussion:

Sequencing of apple genes and analysis of sequence databases has yielded a small number of atypical α-amylase sequences. Apple has at least six distinct α-amylase-like genes, grouped into three families, each containing two genes, while Arabidopsis has only one representative for each family. Apple is a cryptic diploid plant, i.e. it has evolved by the fusion of two ancestral genomes, each with its own set of genes and hence one gene copy from each α-amylase family. Thus we can assume that the three sequence fragments attributed to Mdamy11 are all from a single gene, as the other Family three gene (Mdamy10) has been fully sequenced. We cannot make the same assumptions regarding the sequence fragments from kiwifruit, as we have not sequenced any single family three gene in its entirety.

Perhaps the most striking difference between each gene family is the number of introns within each gene and their relative position. Each of the three families has its own characteristic intron structure, with anywhere from two to twelve introns interrupting the coding sequence; Families one and three do not share any common intro/exon boundaries, although both share boundaries with Family two.

Our investigations have shown that Family three α-amylases are expressed in a variety of different tissues, in a number of plant species. A recent microarray analysis of diurnal and circadian regulated genes in A. thaliana (SCHAFFER et al., 2001) identified an EST from Atamy3 as one of many transcripts displaying a diurnal expression pattern. The Atamy3 transcript is upregulated in the afternoon, along with a hexose transporter, and repressed again in the early morning. This pattern corresponds to the diurnal breakdown of starch in chloroplasts and subsequent export of sugars from photosynthetic tissues, which takes place at night, and is consistent with the predicted plastid-localisation of Atamy3. The expression pattern for Mdamy10 has not been specifically explored, although its presence in many different tissues has been demonstrated by EST sequencing and some initial microarray experiments. Information from a wide range of plants suggests that Family three α-amylases are expressed in plant tissues involved in degradation of plastid-bound starch, including photosynthetic cells (during night-time), fruit during maturation and floral buds breaking in spring.

The 5′ and 3′ untranslated regions of each gene may be important in post-transcriptional regulation, possibly controlling the stability or localisation of the mRNA transcript. The 3′ UTR of a rice α-amylase transcript has been shown to mediate mRNA levels in a sugar-dependent manner, by destabilising the transcript when sugar is abundant (CHAN & YU, 1998). Mdamy10 has a long 3′ UTR that may contain similar mRNA stability elements.

We were able to amplify sequence of family three genes from several different plant species, using degenerate primers designed from the DNA sequences of Atamy3, Mdamy10, and Osamy10. Sequences from coffee, cotton and rose all showed a high degree of identity to Mdamy10, and almost certainly come from family three genes, based on the degree of identity and the position of the introns within each sequence. It is quite likely that the other PCR products shown in FIG. 10, also represent family three α-amylases, and that the family is ubiquitous in plants. The PCR fragments could be used as probes to isolate full-length α-amylase sequences from the source organsims, as well as related species; they could also be used as molecular markers in plant breeding.

Mdamy10 was successfully expressed in E. coli, and has been shown to possess the ability to enzymatically degrade amylopectin in native gels. Unfortunately the activity of the enzyme appears to be lost upon purifying by nickel ion affinity chromatography. The crude protein extract produces two Mdamy10 specific bands when electrophoresed into amylopectin containing gels, but only one band when transferred into amylopectin gels from a native PAGE gel. The lower band seen on starch gels could be caused by partially degraded protein; some evidence for this is seen in western blots using anti-His₆ antibodies, where a smaller (55 kDa) protein is eluted slightly later than the 105 kDa Mdamy10 band. The 55 kDa band may represent the N-terminal half of the Mdamy10 protein, which would retain the His₆ protein tag, and the C-terminal part of the protein may be released but could still remain enzymatically active. The C-terminal fragment would not be purified by nickel ion affinity chromatography, and would not show up on western blots with anti-His₆ antibody. However, loss of this fragment is not the reason for loss of activity during purification, as activity is lost upon desalting on a PD-10 column (which would not remove the fragment). The C-terminal fragment would also run much further than the full-length protein, on a native PAGE gel, and may well have eluted from the gel, along with the intrinsic E. coli activity seen on starch gels.

The lack of a second activity band on starch transfer gels could also be explained if the pool of expressed Mdamy10 protein did not have a uniform affinity for starch. This could be due to misfolding of one or more of the domains of the protein, or due to some chemical alteration made to the protein before, during, or after extraction from E. coli, for example oxidation of amino acid residues critical for binding amylopectin. Alternatively, the Mdamy10 protein may form multimeric complexes in the presence of amylopectin, reducing its mobility in starch gels and producing multiple activity bands. Starch transfer gels also show that there is an intrinsic amylolytic activity in apple leaves that co-migrates with Mdamy10 protein expressed in E. coli.

The ability of Mdamy10 to degrade starch, along with its sequence similarity to previously characterised α-amylase genes, largely confirms that it is an active α-amylase.

Expression of an Mdamy10-GFP fusion protein in plant cells was able to demonstrate that the N-terminal end of the protein contains a plastid targeting signal that is capable of effecting GFP import into the chloroplasts of N. benthamiana cells. This confirms earlier computational predictions of such targeting signals in the protein. The existence of very similar peptide sequences in other Family three proteins indicates that the entire family is plastid targeted. This is significant, as no other α-amylases have been described that are targeted to the plastid, which is the main site of starch storage and degradation within plants.

We believe that the Family three α-amylases presently described (including Mdamy10, Mdamy11, Atamy3 and Osamy10) are the enzymes responsible for degrading all forms of plastid-bound starch, i.e. both diurnal and storage starch.

### INDUSTRIAL APPLICATION

In its primary aspect, the invention has application in modulating the starch content of organisms including plants, and plant plastids. This family of α-amylases is implicated in the modification and degradation of plastid-bound starch, that is, both diurnal and storage starch. The invention can be used to modify various aspects of organisms including plants. Such aspects include, starch content, starch composition, starch polymer type, sugar content, ripening, texture, solids content, viscosity of processed tissue, resistance to chilling damage, processing properties, wood quality and yield.

Examples of commercially valuable processes where the methods of the invention directed at modulating starch degradation in transgenic plants may be useful include:
1) prevention of low temperature sweetening in potato tubers which may require inhibition of starch degradation
2) prevention of tuber sprouting which may require inhibition of starch degradation
3) improvement in the storage of starch-containing fruit such as banana, apple, kiwifruit, papaya and mango, where inhibition of starch degradation may aid storage of the fruit, possibly without a need for low temperature storage.
4) dormancy breaking (and germination of seeds) which requires action of starch degrading enzymes to provide energy for new growth.

Chimeric genetic constructs according to the invention can be used to target chimeric proteins to plastids and/or starch granules for a variety of purposes including biopharming of vaccines, or targeting of proteins to modify, either plastid or non-plastid, starch in transgenic plants. Such genetic constructs may also be used to target bacterial, fungal or algal amylases to the starch granules and/or plastids of transgenic plants. Opportunities for DNA shuffling also exist using the sequences of this invention.

The α-amylases of the invention may of course also be employed in known industrial applications in which starch degradation is required. This includes processing of animal feeds, detergents, food and beverages, textiles, healthcare and brewing.

There are also opportunities to produce the α-amylases of the invention, variants of the enzymes or chimeric proteins including the starch binding domain/motifs by fermentation processes, or recombinant expression, for use in industrial applications. For example genetic constructs of the invention could be used to produce chimeric protein including a fungal amylase and starch binding domain/motifs of the invention for more efficient degradation of plant starch in waste processing.

It will further be appreciated by those persons skilled in the art that the present description is provided by way of example only and that the scope of the invention is not limited thereto.

### REFERENCES

ALTSCHUL, S. F., et al., Gapped BLAST and PSI-BLAST: a new generation of protein database search programs, Nucleic Acids Res. 25: 3389-34023 (1997)
CARVALHO N. et al (Plant Cell 7: 347-258, 1995).
CHAN, M. T. & YU, S. M. 1998. The 3′ untranslated region of a rice α-amylase gene functions as a sugar-dependent mRNA stability determinant. Proc. Natl. Acad. Sci. USA 95: 6543-6547.
CHUA et al. (Science, 244: 174-181, 1989).
DAVIS, S. J. & VIERSTRA, R. D. 1998. Soluble, highly fluorescent variants of green fluorescent protein (GFP) for use in higher plants. Plant Mol. Biol. 36: 521-528.
DUNSTAN et al., Somatic embryogenesis in woody plants. In: Thorpe, T. A. ed. 1995: in vitro embryogenesis of plants. Vol 20 in Current Plant Science and Biotechnology in Agriculture, Chapter 12, pp. 471-540.
EMANUELSSON, O., NIELSEN, H., BRUNAK, S. & VON HEIJNE, G. 2000. Predicting subcellular localization of proteins based on their N-terminal amino acid sequence. J. Mol. Biol. 300: 1005-1016.
EMANUELSSON, O., NIELSEN, H. & VON HEIJNE, G. 1999. ChloroP, a neural network-based method for predicting chloroplast transit peptides and their cleavage sites. Protein Science 8: 978-984.
GLICK & THOMPSON, eds., Methods in Plant Molecular Biology, CRC Press, Boca Raton, Fla. (1993)
BIRCH, R. G., Ann Rev Plant Phys Plant Mol. Biol., 48: 297 (1997);
FORESTER et al., Exp. Agric., 33: 15-33 (1997).
HAWES, C., BOEVINK, P. & MOORE, I. 2000. Green fluorescent protein in plants. In Protein localization by fluorescence microscopy: a practical approach (ed. V. J. Allan), p. 163-177. Oxford University Press.
HUANG, N., SUTLIFF, T. D., LITTS, J. C. & RODRIGUEZ, R. L. 1990. Classification and characterization of the rice α-amylase multigene family. Plant Mol. Biol. 14: 655-668.
LANGENKAMPER, G., MCHALE, R., GARDNER, R. C. & MACRAE, E. A. 1998. Sucrose-phosphate synthase steady-state mRNA increases in ripening kiwifruit. Plant Mol. Biol. 36: 857-869.
LLAVE, C., XIE, Z., KASSCHAU, K. D. & CARRINGTON, J. C. (2002). Cleavage of Scarecrow-like mRNA targets directed by a class of Arabidopsis miRNA. Science. 297: 2053-2056.
LUEHRSEN, K. R., Mol. Gen. Genet. 225: 81-93, 1991)
LUKASHIN, A. & BORODOVSKY, M. 1998. GeneMark.hmm: new solutions for gene finding. Nucleic Acids Res. 26: 1107-1115.
MANIATIS et al., (Molecular Cloning: A Laboratory Manual, Cold Spring Harbour Laboratories, Cold Spring Harbour, N.Y., 1989).
(MCINTYRE CL, MANNERS JM, Transgenic Res. 5(4): 257-262, 1996)
NAPOLI et al. (Plant Cell 2: 279-290, 1990)
NEEDLEMAN & WUNSCH (J. Mol. Biol. 48; 443-453 (1970))
NICHOLAS, K. B. & NICHOLAS, H. B. 1997. GeneDoc: a tool for editing and annotating multiple sequence alignments. Distributed by the author.
W. R. PEARSON, “Rapid and Sensitive Sequence Comparison with FASTP and FASTA, “Methods in Enzymology 183: 63-98 (1990)
W. R. PEARSON & D. J. LIPMAN, “Improved Tools for Biological Sequence Analysis”, Proc. Natl. Acad. Sci. USA 85: 2444-2448 (1988)
ROBINSON-BENION et al., (1995), Anti-sense techniques, Methods in Enzymol. 254(23): 363-375 and Kawasaki et al., (1996), Artific. Organs 20 (8): 836-848.
ROGERS et al., in Methods for Plant Molecular Biology, A Weissbach and H Weissbach eds, Academic Press Inc., San Diego, Calif. (1988)).
SCHAFFER, R., LANDGRAF, J., ACCERBI, M., SIMON, V., LARSON, M. & WISMAN, E. 2001. Microarray analysis of diurnal and circadian-regulated genes in Arabidopsis. Plant Cell 13: 113-123.
THOMPSON, J. D., HIGGINS, D. G. & GIBSON, T. J. 1994. CLUSTALW: improving the sensitivity of progressive multiple sequence alignment through sequence weighting, position-specific gap penalties and weight matrix choice. Nucleic Acids Res. 22: 4673-4680.
WEGRZYN, T., REILLY, K, CIPRIANI, G., MURPHY, P., NEWCOMB, R., GARDNER, R. & MACRAE, E. 2000. A novel α-amylase gene is transiently upregulated during low temperature exposure in apple fruit; Eur. J. Biochem. 267: 1313-1322.
ZUBAY, G. (1973). In vitro synthesis of protein in microbial systems. Annu. Rev. Genet. 7, 267-287.

1. An isolated polypeptide which comprises a plastid alpha-amylase.

2. An isolated polypeptide of claim 1 having a mature sequence derived from a plastid alpha-amylase with an N-terminal extension comprising both a plastid targeting polypeptide and a starch-binding domain.

3. An isolated polypeptide of claim 2 wherein the N-terminal extension comprises:
a) a plastid targeted polypeptide of an amino acid sequence selected from SEQ ID NO: 7, SEQ ID NO: 8, residues 1-70 of SEQ ID NO: 10 and residues 1-53 of SEQ ID NO: 53; or a functionally equivalent variant thereof, and
b) at least one repeat of the defined polypeptide motif pair:
Motif 1 y HW (G/A)y[X]₆₋₉ WXXP[X]₃₋₅PXX(T/S)
Motif 2 F(V/L)y[X]₅₋₈ W[X]₆₋₈(D/N)F

4. An isolated polypeptide of claim 1 having a mature sequence derived from the Mdamy10 amino acid sequence of SEQ ID NO:2 or a functionally equivalent variant thereof.

5. An isolated polypeptide of claim 4 wherein the mature sequence is derived from a sequence with at least 70% identity to the sequence of SEQ ID NO:2.

6. An isolated polypeptide of claim 4 wherein the mature sequence is derived from a sequence with at least 90% identity to the sequence of SEQ ID NO:2.

7. An isolated polypeptide of claim 4 wherein the mature sequence is derived from a sequence with at least 95% identity to the sequence of SEQ ID NO:2.

8. An isolated polypeptide of claim 4 comprising the mature sequence derived from the sequence of SEQ ID NO:2.

9. An isolated polynucleotide encoding a plastid targeted alpha-amylase having the amino acid sequence of SEQ ID NO:2 or a functionally equivalent variant thereof.

10. An isolated polynucleotide of claim 9 wherein the amino acid sequence has at least 70% identity to the sequence of SEQ ID NO:2.

11. An isolated polynucleotide of claim 9 wherein the amino acid sequence has at least 90% identity to the sequence of SEQ ID NO:2.

12. An isolated polynucleotide of claim 9 wherein the amino acid sequence has at least 95% identity to the sequence of SEQ ID NO:2.

13. An isolated polynucleotide of claim 9 encoding the amino acid sequence of SEQ ID NO:2.

14. An isolated polynucleotide having the sequence of SEQ ID NO:1 or a functionally equivalent variant thereof.

15. An isolated polynucleotide of claim 9 wherein the polynucleotide is at least 70% identical in sequence to the nucleic acid sequence of SEQ ID NO:1.

16. An isolated polynucleotide of claim 9 wherein the polynucleotide is at least 90% identical in sequence to the nucleic acid sequence of SEQ ID NO:1.

17. An isolated polynucleotide of claim 9 wherein the polynucleotide is at least 95% identical in sequence to the nucleic acid sequence of SEQ ID NO:1.

18. An isolated polynucleotide of claim 9 comprising all of the sequence of SEQ ID NO:1.

19. An isolated polynucleotide comprising at least 15 contiguous nucleotides of the sequence of SEQ ID NO:1.

20. An isolated polynucleotide comprising the polynucleotide sequence of SEQ ID NO:3.

21. An isolated polynucleotide comprising a sequence which encodes the amino acid sequence of SEQ ID NO:4 or a functionally equivalent variant thereof.

22. An isolated polynucleotide of claim 21 comprising a sequence which encodes the amino acid sequence of SEQ ID NO:4.

23. An isolated polynucleotide which encodes the amino acid sequence of SEQ ID NO:5, or a functionally equivalent variant thereof.

24. An isolated polynucleotide having the polynucleotide sequence of SEQ ID NO:6.

25. An isolated polynucleotide encoding a chimeric protein which encodes a plastid targeting peptide of an amino acid sequence selected from SEQ ID NO:7, SEQ ID NO:8, residues 1-70 of SEQ ID NO:10 and residues 1-53 of SEQ ID NO:53; or a functionally equivalent variant thereof.

26. An isolated polynucleotide encoding a chimeric polypeptide which comprises a polypeptide which comprises at least one repeat, of the defined polypeptide motif pair:
Motif 1: yHWGV[X]₇₋₁₀W(D/E)(Q/I)P(P)[X]₃₋₄P(P)[X]₈A(I/L)XTXL
Motif 2: FV(F/L/V)K[X]₂E[X]₂₋₃W[X]₄₋₆GXDF
where capital letters represent conserved amino acids; letters in parentheses represent partly conserved amino acids; y represents a hydrophobic residue; X represents any amino acid; and [X]₄₋₆ represents a run of 4 to 6 unspecified amino acids.

27. An isolated polynucleotide encoding a chimeric polypeptide which comprises at least one repeat of the defined polypeptide motif pair:
Motif 1: yHW(G/A)y[X]₆₋₉WXXP[X]₃₋₅PXX(T/S)
Motif 2: F(V/L)y[X]₅₋₈W[X]₆₋₈(D/N)F
where capital letters represent conserved amino acids; letters in parentheses represent partly conserved amino acids; y represents a hydrophobic residue; X represents any amino acid; and [X]₄₋₆ represents a run of 4 to 6 unspecified amino acids.

28. A genetic construct comprising a polynucleotide of claim 9.

29. A genetic construct comprising in a 5′-3′ direction
a) a promoter sequence;
b) an open reading frame polynucleotide encoding a polypeptide of claim 1; and
c) a termination sequence.

30. A genetic construct of claim 29 where the encoded polypeptide comprises the amino acid sequence of SEQ ID NO:2.

31. A genetic construct of claim 29 where the open reading frame is in a sense orientation.

32. A genetic construct of claim 29 where the open reading frame is in an anti-sense orientation.

33. A genetic construct comprising in a 5′-3′ direction
a) a promoter sequence;
b) a non-coding region of a gene encoding a polypeptide of claim 1; and
c) a termination sequence.

34. A genetic construct of claim 33 where the polypeptide has an amino acid sequence of SEQ ID NO:2 and the non coding region is in the antisense orientation.

35. A genetic construct comprising in a 5′-3′ direction
a) a promoter sequence;
b) a polynucleotide comprising a polynucleotide sequence complementary to at least 15 residues of a sequence coding for a polypeptide of claim 1; and
c) a termination sequence.

36. A host cell comprising a genetic construct of claim 28.

37. A transgenic plant cell which comprises a genetic construct of claim 28.

38. A method for modulating the starch content of a plant, the method comprising:
increasing or decreasing expression of a polypeptide of claim 1;
wherein said increasing or decreasing is achieved by genetic modification to alter the expression of a gene encoding a plastid targeted alpha amylase.

39. A method as claimed in claim 38 wherein the polypeptide comprises a polypeptide with the sequence of SEQ ID NO:2.

40. A genetic construct comprising a polynucleotide sequence comprising the plastid targeting signal of SEQ ID NO:7.

41. A method for altering starch metabolism in a plant, the method comprising of
a) introducing into the plant, a genetic construct of claim 28; and
b) transcriptionally expressing the polynucleotide in the plant.

42. A method for altering starch metabolism in a plant, the method comprising of
a) introducing into the plant, a DNA genetic construct of claim 28; and
b) expressing the polypeptide in the plant.

43. A genetic construct comprising in a 5′-3′ direction
a) a promoter sequence;
b) a polynucleotide sequence encoding a plastid targeting signal polypeptide of claim 25 or a functionally equivalent variant thereof, translationally fused to an additional polypeptide-encoding polynucleotide; and
c) a termination sequence.

44. A genetic construct of claim 43 in which the polynucleotide sequence, encoding a plastid targeting signal polypeptide, comprises that of SEQ ID NO:6 or a functionally equivalent variant thereof.

45. A genetic construct of claim 43 in which the plastid targeting signal polypeptide of the invention, comprises that of SEQ ID NO: 7 or a functionally equivalent variant thereof.

46. A host cell comprising a genetic construct of claim 43.

47. A transgenic plant cell which comprises a genetic construct of claim 43.

48. A genetic construct comprising a polynucleotide of claim 27 encoding a starch binding domain polypeptide.

49. A genetic construct comprising in a 5′-3′ direction
a) a promoter sequence;
b) a polynucleotide sequence encoding a starch binding domain polypeptide of claim 27, or a functionally equivalent variant thereof, translationally fused to an additional polypeptide-encoding polynucleotide; and a termination sequence.

50. A genetic construct of claim 49 in which the polynucleotide sequence, encoding a starch binding domain polypeptide, comprises a sequence of SEQ ID NO: 5, or functionally equivalent variants thereof.

51. A host cell comprising a genetic construct of claim 48.

52. A transgenic plant cell which comprises a genetic construct of claim 48, as well as a transgenic plant comprising such cells.

53. A plant genetically modified so that it does not express a peptide selected from SEQ ID NO:4, and SEQ NO:5; or a functionally equivalent variant thereof.

54. A method as claimed in claim 41 wherein the genetic construct comprises a polynucleotide selected from SEQ ID NO:1, SEQ ID NO:15 and SEQ ID NO:52, or a functionally equivalent variant thereof.


SEQUENCE LISTING (55 sequences)

SEQ ID NO: 1 - DNA, 3324 bp, Malus domestica
attccgtccatcagtttcgtttcttcagtcgcctgagcggccgttgctcgttccatgtcgacggttaggatagagcccctcctccaccactaccgtcgacagaaacccagccaccgccttccgccgtcgaagcacccattaaagctcagctcttctttcactgcttttccaaagaagctagtagtctccaatggccgcagcttctgcaacttccagcctcccactctcagtgtcagagctgcctccaccgatacagccaccgtcgaggccaccgaattcgccgacgccttctacaaggagaccttccctctcaagcgaactgaagtggtggagggaaagatgatcgtgaaattagataatgggaaggatgcaaagaattgggtgctgactgtgggttgtaatcttcctggaaaatgggttcttcactggggagttaattatgtggatgacgtcggcagtgaatgggatcagcctcctagtgaaatgagaccagctggttcagtttccatcaaggactatgcaatagagacacccttgaaggaatcgttgtcgccggtgggaggcgatacatctcacgaagtgaagattgatgttacacccaacagcgcaatcgcagcgataaattttgttctcaaggatgaagaaaccggtgcctggtatcagcatagagggagagactttaaagtgcctttcgtgggctacctgcaagacgatgacaatgtagttggagcaacaagggccttgggcgcgtggtcaggaactttgggaaaactatccaatgtgtttgtcaaagcagaaacatcaaattccaaagatcaagaaagcagcagtgaatccagagaccctcaacagaaaactatgcgtctagaaggattctatgaagaactgccaattgcaaaagaaattgctgttaaccattcagcaactgtttccgttaggaagtgccctgagactactaagaatcttctatacttggaaacagatttacctgatcatgctgttgttcactggggagtttgcagagatgatgctaaaagatgggaaattccagctgccccgcatccaccagaaacagtagttttcaaggacaaggctttgcggactcgattacagcaaagggaggatggaaatggatgttctggactgtttaccttggaagaaggactcgcaggatttctttttgttttcaaactaaatgaaactatgtggttgaattgtgtgggcaatgacttctacatcccccttttaagctcaaataactcaattgctgtgcaaaatgaggttcagtctgaagatgctcaggtacctgacagaagtagagaaactaattttactgcatataccgatggtataatcaatgaaataaggaacttggtgagtgatatttcctctgagaagagtcaaaggaaaagatccaaagaagcacaagaaaccattcttcaagaaatagaaaaattggctgctgaagcgtatagtatcttcagaactactgttccaactctaccggaggaaatcattgcagaaactgaaaaggtgaaagtcgcccctgccaaaatatgctcggggacaggaacaggttttgaaatattgtgccaaggttttaactgggaatctagtaaatctggaagatggtacgaggaactcaaaagtaaagctgcagaattatcctcactaggtttcactgtgatttggttcccacctcctacagattctgtgtcgcctcaagggtacatgccgagggatttatacaatatgaactccagatatggaaatatggacgaactgaaggagactgtgaagacattccatgatgccggtttaaaagttcttggagatgctgttctgaatcaccgttgtgcagaatatcagaatcaaaatggtgtttggaatatatttggtggtcgtttaaattgggatgaacgtgcagttgttgcagatgatccacattttcagggtaggggcaacaaaagtagtggagatagttttcatgctgccccaaacattgatcattcacaagattttgtgaggaaggatatcagagaatggttatgctggctaagggacgatattgggtatgacggatggaggcttgatttcgttagaggattttggggtggctacgtcaaggactacatggatgccagtgagccctactttgccgtaggcgagtattgggattccctaagttatacatacggggaaatggatcacaatcaagatgcacacaggcagagaattgttgattggatcaacgctactaatggaacttgtggtgcattcgatgtcacaacaaaagggattctccatgcagcactggaaagatgcgagtattggcgattgtcagatgagaagggtaagcctccaggggttcttggatggtggccatctcgtgctgtcactttcatagagaatcatgatactggttctactcagggtcattggaggtttccaaataaaaaagaaatgcaaggatatgcatacattttgactcatccgggaacacctacagttttctatgaccacattttctctcactaccaatctgaaattgctgctctgatctctctcagaaaccggaacaagctcaactgtcggagtagagttaaaatcaccaaggcagaaagagacgtctacgcggccatcattgatgaaaaggtcgccatcaaaatcggaccaggtcattacgaacctgctagtggacctcaaaattggaataaaagccttgagggaagagactacaaggtctgggaagcgtcataaattttttctgcaccggtatgtatgtaacttcgagtgtataaggtttgagtaccggatgattcaataaggaagttcccatatttacattgtaagcaaatcaaaggatgccgagatagagtaatatgtacttctctctagaggtctggctgacacccttgaattgaagttgtttcggcagacattttccgaatttaagggactgtagacgctcaactgaggacgagacgggccagcctgcttcagcagcacagaatcgcggccagtcggctgagtgtggaagtggtgagcatggtactgggttccaagggcagtgctccatcttacacacactgttgactctccaacttatactgatgcttttaaccaataatgtataaattggtgcatgccagtgactcgtggtccatgaacgttgagtgagtttcctatctgaacttttattctgtctcacattgctccagtccatcaactttatgcatggactcgttaattttttagtattgttcttgaaacaggatgatcgatatatcgttaatatgtgatccatatattatattaaaaaaa

SEQ ID NO: 2 - protein, 901 aa, Malus domestica
MSTVRIEPLLHHYRRQKPSHRLPPSKHPLKLSSSFTAFPKKLVVSNGRSFCNFQPPTLSVRAASTDTATVEATEFADAFYKETFPLKRTEVVEGKMIVKLDNGKDAKNWVLTVGCNLPGKWVLHWGVNYVDDVGSEWDQPPSEMRPAGSVSIKDYAIETPLKESLSPVGGDTSHEVKIDVTPNSAIAAINFVLKDEETGAWYQHRGRDFKVPFVGYLQDDDNVVGATRALGAWSGTLGKLSNVFVKAETSNSKDQESSSESRDPQQKTMRLEGFYEELPIAKEIAVNHSATVSVRKCPETTKNLLYLETDLPDHAVVHWGVCRDDAKRWEIPAAPHPPETVVFKDKALRTRLQQREDGNGCSGLFTLEEGLAGFLFVFKLNETMWLNCVGNDFYIPLLSSNNSIAVQNEVQSEDAQVPDRSRETNFTAYTDGIINEIRNLVSDISSEKSQRKRSKEAQETILQEIEKLAAEAYSIFRTTVPTLPEEIIAETEKVKVAPAKICSGTGTGFEILCQGFNWESSKSGRWYEELKSKAAELSSLGFTVIWFPPPTDSVSPQGYMPRDLYNMNSRYGNMDELKETVKTFHDAGLKVLGDAVLNHRCAEYQNQNGVWNIFGGRLNWDERAVVADDPHFQGRGNKSSGDSFHAAPNIDHSQDFVRKDIREWLCWLRDDIGYDGWRLDFVRGFWGGYVKDYMDASEPYFAVGEYWDSLSYTYGEMDHNQDAHRQRIVDWINATNGTCGAFDVTTKGILHAALERCEYWRLSDEKGKPPGVLGWWPSRAVTFIENHDTGSTQGHWRFPNKKEMQGYAYILTHPGTPTVFYDHIFSHYQSEIAALISLRNRNKLNCRSRVKITKAERDVYAAIIDEKVAIKIGPGHYEPASGPQNWNKSLEGRDYKVWEAS

SEQ ID NO: 3 - DNA, 1468 bp, Malus domestica
agcggccgttgctcgttccatgtcgacggttaggatagagcccctcctccaccactaccgtcgacagaaacccagccaccgccttccgccgtcgaagcacccattaaagctcagctcttctttcactgcttttccaaagaagctagtagtctccaatggccgcagcttctgcaacttccagcctcccactctcagtgtcagagctgcctccaccgatacagccaccgtcgaggccaccgaattcgccgacgccttctacaaggagaccttccctctcaagcgaactgaagtggtggagggaaagatgatcgtgaaattagataatgggaaggatgcaaagaattgggtgctgactgtgggttgtaatcttcctggaaaatgggttcttcactggggagttaattatgtggatgacgtcggcagtgaatgggatcagcctcctagtgaaatgagaccagctggttcagtttccatcaaggactatgcaatagagacacccttgaaggaatcgttgtcgccggtgggaggcgatacatctcacgaagtgaagattgatgttacacccaacagcgcaatcgcagcgataaattttgttctcaaggatgaagaaaccggtgcctggtatcagcatagagggagagactttaaagtgcctttcgtgggctacctgcaagacgatgacaatgtagttggagcaacaagggccttgggcgcgtggtcaggaactttgggaaaactatccaatgtgtttgtcaaagcagaaacatcaaattccaaagatcaagaaagcagcagtgaatccagagaccctcaacagaaaactatgcgtctagaaggattctatgaagaactgccaattgcaaaagaaattgctgttaaccattcagcaactgtttccgttaggaagtgccctgagactactaagaatcttctatacttggaaacagatttacctgatcatgctgttgttcactggggagtttgcagagatgatgctaaaagatgggaaattccagctgccccgcatccaccagaaacagtagttttcaaggacaaggctttgcggactcgattacagcaaarggaggatggaaatggatgttctggactgtttaccttgraagaaggactcgcaggatttctttttgttttcaaactaaatgaamytatgtggttgaattgtgtgggcaatgacttctacatcccccttttaagctcaaataactcaattgctgtgcaaaatgaggttcagtctgaagatgctcaggtacctgmcagaagtagagaaactaattttactgcatataccgatggtataatcaatgaaataaggaacttggtgagtgatatttcctctgagaagagtcaaaggaaaagatccaaagaagcacaagaaaccattcttcaagaaatagaaaaattggctgctgaagcgtatagtatcttcagaactactgttccaactcta

SEQ ID NO: 4 - protein, 483 aa, Malus domestica
  Unknown (1)..(483): X is any amino acid.
MSTVRIEPLLHHYRRQKPSHRLPPSKHPLKLSSSFTAFPKKLVVSNGRSFCNFQPPTLSVRAASTDTATVEATEFADAFYKETFPLKRTEVVEGKMIVKLDNGKDAKNWVLTVGCNLPGKWVLHWGVNYVDDVGSEWDQPPSEMRPAGSVSIKDYAIETPLKESLSPVGGDTSHEVKIDVTPNSAIAAINFVLKDEETGAWYQHRGRDFKVPFVGYLQDDDNVVGATRALGAWSGTLGKLSNVFVKAETSNSKDQESSSESRDPQQKTMRLEGFYEELPIAKEIAVNHSATVSVRKCPETTKNLLYLETDLPDHAVVHWGVCRDDAKRWEIPAAPHPPETVVFKDKALRTRLQQXEDGNGCSGLFTLXEGLAGFLFVFKLNEXMWLNCVGNDFYIPLLSSNNSIAVQNEVQSEDAQVPXRSRETNFTAYTDGIINEIRNLVSDISSEKSQRKRSKEAQETILQEIEKLAAEAYSIFRTTVPTL

SEQ ID NO: 5 - protein, 265 aa, Malus domestica
  Unknown (1)..(265): X is any amino acid.
WVLHWGVNYVDDVGSEWDQPPSEMRPAGSVSIKDYAIETPLKESLSPVGGDTSHEVKIDVTPNSAIAAINFVLKDEETGAWYQHRGRDFKVPFVGYLQDDDNVVGATRALGAWSGTLGKLSNVFVKAETSNSKDQESSSESRDPQQKTMRLEGFYEELPIAKEIAVNHSATVSVRKCPETTKNLLYLETDLPDHAVVHWGVCRDDAKRWEIPAAPHPPETVVFKDKALRTRLQQXEDGNGCSGLFTLXEGLAGFLFVFKLNEXMW

SEQ ID NO: 6 - DNA, 183 bp, Malus domestica
atgtcgacggttaggatagagcccctcctccaccactaccgtcgacagaaacccagccaccgccttccgccgtcgaagcacccattaaagctcagctcttctttcactgcttttccaaagaagctagtagtctccaatggccgcagcttctgcaacttccagcctcccactctcagtgtcaga

SEQ ID NO: 7 - protein, 61 aa, Malus domestica
MSTVRIEPLLHHYRRQKPSHRLPPSKHPLKLSSSFTAFPKKLVVSNGRSFCNFQPPTLSVR

SEQ ID NO: 8 - protein, 55 aa, Arabidopsis thaliana
MSTVPIESLLHHSYLRHNSKVNRGNRSFIPISLNLRSHFTSNKLLHSIGKSVGVS

SEQ ID NO: 9 - DNA, 934 bp, Malus domestica
gttctcagtctmccgagtggccgttgctcataccatgtcgacggttaggatagagcccctcctccaggacctccaccactacggtcgacaaaaacccagccaccgccgtccgcagtcgaatcatccattaaagctcagctcttctttcactgcttttcctaagaagctagtagtctccaacagccgcagcttctgttacttccagcctcccactcccaggaggggtcccactctcggagtcagagctgcctccaccgatacaaccactgtcgagacctccgaatccaccgatcccatctacaagaagaccttccctctcaagcggacggaagtggtggagggaaagatctttgtgaaattagatcatgggaagaatgaaaagaagtgggtgctgactgtgggttgtaatcttcctggaaaatgggttcttcattggggagtttcttttgtggacgatgttagttgcgaatgggaacagcctcctagtgaaatgagaccagctggttcagttcccatcaaggactatgcaatagagacacccttgaaggaatcgttgtcgtctgtgggaggcgatacatcttatgaagtgaagattgatgttaaacccaacagcgcaattgcagcgataaattttgttctcaaggatgaagaaactggtgcttggtatcagcacagagggagtgactttagagtgcctcttgtcgcctacccccaagacgatgacaatgtagttggagcgacaaagggctttggcatgtggccaggttgtcaatgccactctattctctggtttgcttgatttattttgtttgtaatactgktaaatttgaaccaatttgtgattttccaagaaacggtgtaataaaagcccaaaaggrgmctttcctaattcagcttaaaaaaaaaaaaaaaaaaaaaaaaaaaaaaaaaaaaa

SEQ ID NO: 10 - protein, 254 aa, Malus domestica
MSTVRIEPLLQDLHHYGRQKPSHRRPQSNHPLKLSSSFTAFPKKLVVSNSRSFCYFQPPTPRRGPTLGVRAASTDTTTVETSESTDPIYKKTFPLKRTEVVEGKIFVKLDHGKNEKKWVLTVGCNLPGKWVLHWGVSFVDDVSCEWEQPPSEMRPAGSVPIKDYAIETPLKESLSSVGGDTSYEVKIDVKPNSAIAAINFVLKDEETGAWYQHRGSDFRVPLVAYPQDDDNVVGATKGFGMWPGCQCHSILWFA

SEQ ID NO: 11 - DNA, 519 bp, Malus domestica
gaaactaaatgaaagtacgtggttgagatgtgtgggaaatgacttctacatccccctttcaagctcaaaaaacgcaaatgttgtacaatcagagattcaatctaaagatgctcaggtacctgacgggagtacagaagcagtagaagaaagtactgcatatgccgatggattaatcaatgaaatgaggaacttggtgagtgacgttttctctgataaaagtccaaggacaagatccaaaaaagcacaagaagccattcttcaagaaatagaaaaattggctgctgaagcgtatagtatcttcagaactaccgttccaactttacctgaggaaaccattgcagaaactgaagaagtgaaggtcgcccctgccaaaatatgttcagggacaggaacaggtttcgaaatattgtgccaaggttttaactgggaatctagtaaatctggaagatggtacatggaactcaaaagtaaagctgcactgttatcttcactaggtttcactgtgatttggttcccacc

SEQ ID NO: 12 - protein, 172 aa, Malus domestica
KLNESTWLRCVGNDFYIPLSSSKNANVVQSEIQSKDAQVPDGSTEAVEESTAYADGLINEMRNLVSDVFSDKSPRTRSKKAQEAILQEIEKLAAEAYSIFRTTVPTLPEETIAETEEVKVAPAKICSGTGTGFEILCQGFNWESSKSGRWYMELKSKAALLSSLGFTVIWFP

SEQ ID NO: 13 - DNA, 426 bp, Malus domestica
gatatcagagaatggttatgctggctaaggaacgatattgggtacgatggatggaggctggattttgttagaggatttcggggtggctatgtcagggactacgtggatgctagtgagccctactttgctgtaggcgagtattgggattccctaagttatacgtatggggaaatggatcgcaatcaagatgcacacaggcagagaattgttgattggatcaacgctactaatggaacttgtggtgcatttgatgtcacaacaaaagggattctccatgcagcgctggaaaggtgcgagtattggcgattgtcagatgagaagggtaagcctccaggggttcttggatggtggccatctcgtgctgccactttcatagagaatcatgatactggttctactcagggtcattggaggtttccaaataaa

SEQ ID NO: 14 - protein, 142 aa, Malus domestica
DIREWLCWLRNDIGYDGWRLDFVRGFRGGYVRDYVDASEPYFAVGEYWDSLSYTYGEMDRNQDAHRQRIVDWINATNGTCGAFDVTTKGILHAALERCEYWRLSDEKGKPPGVLGWWPSRAATFIENHDTGSTQGHWRFPNK

SEQ ID NO: 15 - DNA, 2664 bp, Arabidopsis thaliana
atgtccactgttcccattgagtctcttctccaccattcttatcttcgtcacaactcaaaagtcaatcgcggaaatagaagcttcatacctatctcgttgaatctccgttctcatttcacttctaacaaactactacactcaattggaaaaagtgtcggtgttagctcgatgaacaaaagtcccgtcgccattcgtgctacctcatcggatactgccgtcgtggaaaccgctcaatcggatgatgtcatcttcaaggaaattttccctgttcagcgaatcgaaaaggcagaaggaaagatttatgttcgattaaaggaagtgaaggagaagaattgggagctgagtgttggatgtagtatacctggaaaatggattttgcattggggagtttcatatgtgggtgatactggcagtgaatgggatcaacctccggaagatatgagacctcctggctcaattgccattaaggactatgccatagagacacctttgaagaagttatctgaaggagattctttttttgaagttgctattaatctaaatctggagagttctgtagcagctctcaattttgttttgaaggacgaagaaactggggcgtggtatcagcacaaagggagagactttaaggttcctcttgtagacgatgttcctgataatggaaatttgatcggagctaagaaaggatttggtgcccttgggcagctttcaaacatccctctcaaacaagataagtccagtgcagagactgattctattgaagaaagaaaaggtcttcaagagttttacgaggagatgccaataagtaaacgtgttgccgatgataactcagtcagtgtcactgcaaggaaatgccctgaaacatctaagaacattgtatcaattgaaaccgatttaccgggtgacgttactgtccactggggagtttgcaaaaacggcactaagaaatgggaaattccatctgaaccttaccctgaagagacatctttgtttaagaacaaggcattacgcactcgtttacagcgaaaagacgatggaaatggatcgtttggattattctctctggatggaaagcttgaagggttatgctttgttcttaagttaaatgaaaatacttggctaaattataggggggaagacttctatgtccctttccttacttcaagtagctcgcccgttgaaactgaagctgcccaagtgagtaaacccaaacgaaaaacagataaagaagtgtctgctagtggatttactaaagaaatcatcacggagataaggaacttggcaattgacatttcctctcataagaatcagaagacaaacgtcaaagaagtgcaggaaaacattctacaagaaattgagaaactggctgcggaggcatatagcatatttagaagcacaactccagctttttccgaggaaggtgttttagaagcagaagctgacaagcctgacattaaaatctcctcaggaaccggctcgggatttgagatattatgccaaggtttcaactgggagtccaataaatctgggagatggtacttggaacttcaagaaaaagccgatgagttagcttcacttggattcactgttctgtggttacctccaccgacagaatctgtgtcacctgaagggtacatgcctaaagacctgtataacttgaattccagatatggaacaattgatgagctaaaagatacagtgaagaaatttcacaaggtcgggatcaaagttttaggcgatgctgttctgaatcaccgctgtgcacacttcaaaaatcaaaatggtgtatggaatctatttggaggacgtctaaactgggacgatagggcagtagttgcagatgaccctcatttccagggtagaggaaacaagagcagtggagataatttccatgctgctccaaacatagatcactcgcaagactttgttaggaaggatatcaaggaatggctatgctggatgatggaagaagttgggtatgatggatggaggcttgactttgtaagagggttctggggaggttatgtgaaagactatatggatgctagcaaaccgtactttgcggttggtgaatattgggattcgttaagttacacgtacggagaaatggactacaatcaagacgcacatcgtcaaagaatagttgactggattaatgcaactagtggagctgctggtgcatttgatgtcactaccaaaggaattcttcatacggcgcttcaaaaatgtgaatattggagactctcagacccaaaagggaagcctccaggtgtagttggttggtggccttctcgtgctgtaacattcatcgagaatcatgacactggctctacacagggtcattggagatttccggaggggaaggaaatgcaaggatatgcttacatcctaactcatccaggaacaccagcggtcttcttcgaccatatcttctcggattatcattccgagattgctgcacttctctctctcaggaacagacagaaactccactgtcggagtgaggtgaatatagacaagagtgagagagatgtgtatgcggctataatagatgaaaaggttgcaatgaagatcggaccagggcattatgaaccaccaaacggatcgcaaaactggtctgtagccgttgaaggcagagactacaaggtgtgggaaacatcttaa

SEQ ID NO: 16 - DNA, 2939 bp, Arabidopsis thaliana
aaaatatcttcttcttctttcttctgctctctctctctctctcgccatgtccactgttcccattgagtctcttctccaccattcttatcttcgtcacaactcaaaagtcaatcgcggaaatagaagcttcatacctatctcgttgaatctccgttctcatttcacttctaacaaactactacactcaattggaaaaagtgtcggtgttagctcgatgaacaaaagtcccgtcgccattcgtgctacctcatcggatactgccgtcgtggaaaccgctcaatcggatgatgtcatcttcaaggaaattttccctgttcagcgaatcgaaaaggcagaaggaaagatttatgttcgattaaaggaagtgaaggagaagaattgggagctgagtgttggatgtagtatacctggaaaatggattttgcattggggagtttcatatgtgggtgatactggcagtgaatgggatcaacctccggaagatatgagacctcctggctcaattgccattaaggactatgccatagagacacctttgaagaagttatctgaaggagattctttttttgaagttgctattaatctaaatctggagagttctgtagcagctctcaattttgttttgaaggacgaagaaactggggcgtggtatcagcacaaagggagagactttaaggttcctcttgtagacgatgttcctgataatggaaatttgatcggagctaagaaaggatttggtgcccttgggcagctttcaaacatccctctcaaacaagataagtccagtgcagagactgattctattgaagaaagaaaaggtcttcaagagttttacgaggagatgccaataagtaaacgtgttgccgatgataactcagtcagtgtcactgcaaggaaatgccctgaaacatctaagaacattgtatcaattgaaaccgatttaccgggtgacgttactgtccactggggagtttgcaaaaacggcactaagaaatgggaaattccatctgaaccttaccctgaagagacatctttgtttaagaacaaggcattacgcactcgtttacagcgaaaagacgatggaaatggatcgtttggattattctctctggatggaaagcttgaagggttatgctttgttcttaagttaaatgaaaatacttggctaaattataggggggaagacttctatgtccctttccttacttcaagtagctcgcccgttgaaactgaagctgcccaagtgagtaaacccaaacgaaaaacagataaagaagtgtctgctagtggatttactaaagaaatcatcacggagataaggaacttggcaattgacatttcctctcataagaatcagaagacaaacgtcaaagaagtgcaggaaaacattctacaagaaattgagaaactggctgcggaggcatatagcatatttagaagcacaactccagctttttccgaggaaggtgttttagaagcagaagctgacaagcctgacattaaaatctcctcaggaaccggctcgggatttgagatattatgccaaggtttcaactgggagtccaataaatctgggagatggtacttggaacttcaagaaaaagccgatgagttagcttcacttggattcactgttctgtggttacctccaccgacagaatctgtgtcacctgaagggtacatgcctaaagacctgtataacttgaattccagatatggaacaattgatgagctaaaagatacagtgaagaaatttcacaaggtcgggatcaaagttttaggcgatgctgttctgaatcaccgctgtgcacacttcaaaaatcaaaatggtgtatggaatctatttggaggacgtctaaactgggacgatagggcagtagttgcagatgaccctcatttccagggtagaggaaacaagagcagtggagataatttccatgctgctccaaacatagatcactcgcaagactttgttaggaaggatatcaaggaatggctatgctggatgatggaagaagttgggtatgatggatggaggcttgactttgtaagagggttctggggaggttatgtgaaagactatatggatgctagcaaaccgtactttgcggttggtgaatattgggattcgttaagttacacgtacggagaaatggactacaatcaagacgcacatcgtcaaagaatagttgactggattaatgcaactagtggagctgctggtgcatttgatgtcactaccaaaggaattcttcatacggcgcttcaaaaatgtgaatattggagactctcagacccaaaagggaagcctccaggtgtagttggttggtggccttctcgtgctgtaacattcatcgagaatcatgacactggctctacacagggtcattggagatttccggaggggaaggaaatgcaaggatatgcttacatcctaactcatccaggaacaccagcggtcttcttcgaccatatcttctcggattatcattccgagattgctgcacttctctctctcaggaacagacagaaactccactgtcggagtgaggtgaatatagacaagagtgagagagatgtgtatgcggctataatagatgaaaaggttgcaatgaagatcggaccagggcattatgaaccaccaaacggatcgcaaaactggtctgtagccgttgaaggcagagactacaaggtgtgggaaacatcttaagaagaaggttcccaatttttatgtgtatctagtagtaaataaaattgttcctggaggatatattatttcagggaataagattacctggaaatgttatatttagagtggaattaaaaccagatctaagatttgtcccacttttttcaatgctgagagaatattcatattgttgtattggatgcttgtaaagtgtatgcatatatatatgaataataatatatctatttcc

SEQ ID NO: 17 - protein, 887 aa, Arabidopsis thaliana
MSTVPIESLLHHSYLRHNSKVNRGNRSFIPISLNLRSHFTSNKLLHSIGKSVGVSSMNKSPVAIRATSSDTAVVETAQSDDVIFKEIFPVQRIEKAEGKIYVRLKEVKEKNWELSVGCSIPGKWILHWGVSYVGDTGSEWDQPPEDMRPPGSIAIKDYAIETPLKKLSEGDSFFEVAINLNLESSVAALNFVLKDEETGAWYQHKGRDFKVPLVDDVPDNGNLIGAKKGFGALGQLSNIPLKQDKSSAETDSIEERKGLQEFYEEMPISKRVADDNSVSVTARKCPETSKNIVSIETDLPGDVTVHWGVCKNGTKKWEIPSEPYPEETSLFKNKALRTRLQRKDDGNGSFGLFSLDGKLEGLCFVLKLNENTWLNYRGEDFYVPFLTSSSSPVETEAAQVSKPKRKTDKEVSASGFTKEIITEIRNLAIDISSHKNQKTNVKEVQENILQEIEKLAAEAYSIFRSTTPAFSEEGVLEAEADKPDIKISSGTGSGFEILCQGFNWESNKSGRWYLELQEKADELASLGFTVLWLPPPTESVSPEGYMPKDLYNLNSRYGTIDELKDTVKKFHKVGIKVLGDAVLNHRCAHFKNQNGVWNLFGGRLNWDDRAVVADDPHFQGRGNKSSGDNFHAAPNIDHSQDFVRKDIKEWLCWMMEEVGYDGWRLDFVRGFWGGYVKDYMDASKPYFAVGEYWDSLSYTYGEMDYNQDAHRQRIVDWINATSGAAGAFDVTTKGILHTALQKCEYWRLSDPKGKPPGVVGWWPSRAVTFIENHDTGSTQGHWRFPEGKEMQGYAYILTHPGTPAVFFDHIFSDYHSEIAALLSLRNRQKLHCRSEVNIDKSERDVYAAIIDEKVAMKIGPGHYEPPNGSQNWSVAVEGRDYKVWETS

SEQ ID NO: 18 - DNA, 1030 bp, Malus domestica
aggatatcagagaatggttatgctggctaaggtgaatttctaagatgttctgattttttacaagttcttcaaatttgtctcactcaataaaaacaaagatcgtacgtaaaagagcaaagcacgtactatatcctaaacttgagttattttgcactggaaattatagagattatagattttctgggatttacatggaagtttacctagccttgggatttcaaaatttattgtgatgaggttgtgttcatgcatttcttatgcacattctgacgtagaatatcagtttgtggacgtgcccttacaaaaattcatggaccatgttatgtgtaatcccaattgacgtgcatttcccttttgctgcaatatttttgttgaaagtttactatgttactttgatataaatgtatgagtgatcgtcacagggacgatattgggtatgacggatggaggcttgatttcgttagaggattttggggtggctacgtcaaggactacatggatgccagtgagccctactttgccgtaggcgagtattgggattccctaagttatacatacggggaaatggatcacaatcaagatgcacacaggcagagaattgttgattggatcaacgctactaatggaacttgtggtgcattcgatgtcacaacaaaagggattctccatgcagtaagtgtgcatctttgtaatttaaaactcaactgttaaaacttttatgaccttgctttccatgtttgtatggcggatgatacttttattcacatggaactacttatacaggcactggaaagatgcgagtattggcgattgtcagatacgaagggtaagcctccaggggttcttggatggtggccatctcgtgctgtcactttcatagagaatcatgatactggttctactcaggtttgaatttttttgatcttgaattctgtcattttacaaacaagcatagttatatttacaagtatcgagatacaaattcttttattcttcccttttgaagggtcattggaggtttccaaataaa

SEQ ID NO: 19 - DNA, 831 bp, Malus domestica
gatatcagagaatggttatgctggctaaggttaattcctaacatgttcttatgcccattatgacgtagaatatcaatttgtggacgtaccagtaccttgcaaaaattttggacatgttgtattttatccaattgaagtgcaatctcccttatggtgcatgttatgttgaaagcttatctattatgttgatataaatgtatgactgaccgtcacaggaacgatattgggtacgatggatggaggctggattttgttagaggatttcggggtggctatgtcagggactacgtggatgctagtgagccctactttgctgtaggcgagtattgggattccctaagttatacgtatggggaaatggatcgcaatcaagatgcacacaggcagagaattgttgattggatcaacgctactaatggaacttgtggtgcatttgatgtcacaacaaaagggattctccatgcagtaagtgtgcatctttgtaatttttaattgcaactgtaaaacttctatgactttgctttccatgtttgtatggcggatgctacttttattcacatggaattacttgtacttgtacaggcgctggaaaggtgcgagtattggcgattgtcagatgagaagggtaagcctccaggggttcttggatggtggccatctcgtgctgccactttcatagagaatcatgatactggttctactcaggtttgaactgttttgatcatgaattctctgttattttatagtcaagcatattaatatttaaaagtatcgagatacaaattcttttattctttctttttgaagggtcattggaggtttccaaataaa

SEQ ID NO: 20 - DNA, 667 bp, Actinidia chinensis
ccccgcgtccgcttcagacctcctcaacctctctctgtcagagcgagctccgccgatacagccgtggtggagacctctgactctgtggacgtcttattcaaggagacattcgctttgaagcgaattgaaaaggtggagggacatatatcaatcaagttagataatgggaaagagagagaaaattggcagctttctgtgggatgtaatcttccggggaagtgggttcttcactggggcgtaaactatatcaacgacattggcagtgaatgggatcagcctcctgttgagatgaggcctccaggctctgttcctattaaggattatgcaattgaaactcctctgaagaaatcatctgcagtggtggaaggagatttatattacgaattgaagattgattttagtacggacaaagatattgcagctataaattttgttttgaaggatgaggaaactggagcttggtatcagcgccgaggaagagatttcaaagttgctctcattgacgaccttcatgaagatggcaataaattaggagctaaaaagggcttaggtgtacggccagggccttttgaacagctctctagcctactgctcaaatcagaagaagctcatcccaagggtgaagacaacagtgactctcgaggtcctagtaaaaaaactaagtgcc

SEQ ID NO: 21 - protein, 222 aa, Actinidia chinensis
PRVRFRPPQPLSVRASSADTAVVETSDSVDVLFKETFALKRIEKVEGHISIKLDNGKERENWQLSVGCNLPGKWVLHWGVNYINDIGSEWDQPPVEMRPPGSVPIKDYAIETPLKKSSAVVEGDLYYELKIDFSTDKDIAAINFVLKDEETGAWYQRRGRDFKVALIDDLHEDGNKLGAKKGLGVRPGPFEQLSSLLLKSEEAHPKGEDNSDSRGPSKKTKC

SEQ ID NO: 22 - DNA, 602 bp, Actinidia chinensis
aaaatgggagattcctgccaagccatatcctgctgaaacaatagttttcaagaataaggcattgcggactctattgcagcgaaaagagggtggaaagggtggttggagtttatttactttggatgaaggatatgctggatttgtttttgtgctcaagataaatgaaaacacatggttgaattatatgggaaatgacttttacatacctctttcaagttcaagtgtcttgcctgctcaacctagacatgatcaatctgaaggtcacaggcaggtagagacagatcaagaagtttctcctgctgcatatactgatggaatcatcaatgatataagaagtttagtgagcgatatttcctctgggaagagtcgacaaacgaaatcaaaagaatctcaacaaagcattcttcaagaaattgagaagctggctgcagaagcctacagcatcttcagaagctctattcctacttattcggaggatgtgatggtggaatctgaggaagtagaaccccctgcaaaaatatcttcaggaacaggttctgggtttgaaattctctgtcaaggatttaactgggagtcccataaatctggaagatggtacatgc

SEQ ID NO: 23 - protein, 200 aa, Actinidia chinensis
KWEIPAKPYPAETIVFKNKALRTLLQRKEGGKGGWSLFTLDEGYAGFVFVLKINENTWLNYMGNDFYIPLSSSSVLPAQPRHDQSEGHRQVETDQEVSPAAYTDGIINDIRSLVSDISSGKSRQTKSKESQQSILQEIEKLAAEAYSIFRSSIPTYSEDVMVESEEVEPPAKISSGTGSGFEILCQGFNWESHKSGRWYM

SEQ ID NO: 24 - DNA, 1451 bp, Actinidia chinensis
ttgttgcggatgatccacatttccagggaaggggcaacaagagtagcggtgataatttccatgctgctccaaatattgatcattctcaagaatttgtgaggagagatcttaaagaatggctttgttggctaaggaaagaaattgggtatgatggatggaggcttgattttgttcggggattttggggaggttatatcaaggaytacatagatgcaagtgagccttactttgctgtaggcgagtattgggattctctcagctacacttatggtgagatggatcacaatcaagatgctcataggcagagaattattgaatggatcaatgctactagtggaactgctggtgcatttgacgtcacaactaarggaattctacattctgcgcttcaaagatgcgagtattggcgattatcagatcagaagggaaaacctccaggagttgttgggtggtggccgtctcgggcagttacgtttatagaaaatcatgacactgggtctactcagggtcattggagatttccaggtggaaaggagatgcaagggtatgcatacatcctgactcatcccggaacaccagcagttttctatgatcacgctttccatcgcatgcgatctgaaatttcggcactcgtttctttgagaaaccggaacaagatccactgtcgtagtacaattcaaataaccaaggcagaaagggatgtttatgcagccattatcgacaaaaaggtggcaatgaagataggcccaggtttctacgaacccgcaagtgggccccaaagatggtctctggccgttgagggaaacgattacaaagtctgggaagcgtcatagaatttgaaaatgcattgtcttgaagcacatgaagtttatagaacatcatttcgaccaaaggagagatacaaaatgatattatctataataaagttgaacccttcccccaacggcatacttcaattgaagaccgcattgcagcacatgttcaccttgtttgaggaagtgatcatgtccacgtgatgatatgtgggaattgattggggtttaagtagaaatatacataatacatacttttgtaatcttacacttatattgaattaccagtgtaaactcttgaacatctggaaaataaattcccaagaggcaagagggtattgtaagaactcggacagacagtgtaaaactgttctataaatttgtacgatagtccaaataagctcattatattataaaggtgagttatctacaaaccattctccattgattattgtgtttgcacagtgaaacgagatgatatggaaatctttgaaactaacactgagaagtctgatgtcgactgaatcctctgttgtgatttctgctttgtacgtggagtttttaaggcttcttaaaaggattggattgtcattcaattaagtgacggaaagagactatgtgacctcatggaaagaagcc

SEQ ID NO: 25 - protein, 276 aa, Actinidia chinensis
VADDPHFQGRGNKSSGDNFHAAPNIDHSQEFVRRDLKEWLCWLRKEIGYDGWRLDFVRGFWGGYIKDYIDASEPYFAVGEYWDSLSYTYGEMDHNQDAHRQRIIEWINATSGTAGAFDVTTKGILHSALQRCEYWRLSDQKGKPPGVVGWWPSRAVTFIENHDTGSTQGHWRFPGGKEMQGYAYILTHPGTPAVFYDHAFHRMRSEISALVSLRNRNKIHCRSTIQITKAERDVYAAIIDKKVAMKIGPGFYEPASGPQRWSLAVEGNDYKVWEAS

SEQ ID NO: 26 - DNA, 450 bp, Actinidia chinensis
ggcttctctggtgtatatgtgaaagaatatatagaagcttcaaatcctgcttttgcaattggagaatattgggatagtttggcttacgagggtggtgatttgtgttacaaccaagatgctcatcgacaacggattgttaattggatcaatgccactggcggtgcctcatcagcatttgacgtcacaacaaagggtatattacattctgctctgcataatcaatattggaggttgattgatcctcaaggaaaaccaacaggggtaatggggtggtggccatcgcgtgctgtcacattcttagagaaccatgatacaggatctacacagggtcattggccatttccaagagacaagcttgcacagggatacgcttacattttaacccatccgggaacgcctgtaatattttatgaccatttctatgactttgggctccacgacgtcataacc

SEQ ID NO: 27 - protein, 150 aa, Actinidia chinensis
GFSGVYVKEYIEASNPAFAIGEYWDSLAYEGGDLCYNQDAHRQRIVNWINATGGASSAFDVTTKGILHSALHNQYWRLIDPQGKPTGVMGWWPSRAVTFLENHDTGSTQGHWPFPRDKLAQGYAYILTHPGTPVIFYDHFYDFGLHDVIT

SEQ ID NO: 28 - DNA, 592 bp, Vaccinium corymbosum
tccgaggcattaaaacagcccccacaaataagttcgggaacaggttcaggttttgaaattctgtgccaaggatttaactgggagtctcataaatccggaagatggtacatggagctcaaagaaaaaattgctgaaatgtcttctcttggtttcactgtggtttggttaccaccaccgacagattctgtctcgcctgaaggctacatgccaagggatttgtatgacttgaactccagatacggtggtactgatgagctgaaggttctggtgaagagattccatgaagttggtatcaaagttcttggagatgttgtcctaaatcaccgttgcgcacaatataagaatcaaaatggtgtatggaatattttcggtggtcgtctaaattgggatgatcgtgctgttgttgcagatgatccacatttccagggaagaggcaacaagagtagtggagataatttccatgctgcaccaaatatagatcattcgcaggaatttgtgaggaaggatcttaaaaaatggctacgctggctaaggaaaaatattgggtatgatggatggaggcttgatttcgctcggggattctgggggggtt

SEQ ID NO: 29 - protein, 197 aa, Vaccinium corymbosum
SEALKQPPQISSGTGSGFEILCQGFNWESHKSGRWYMELKEKIAEMSSLGFTVVWLPPPTDSVSPEGYMPRDLYDLNSRYGGTDELKVLVKRFHEVGIKVLGDVVLNHRCAQYKNQNGVWNIFGGRLNWDDRAVVADDPHFQGRGNKSSGDNFHAAPNIDHSQEFVRKDLKKWLRWLRKNIGYDGWRLDFARGFWGG

SEQ ID NO: 30 - DNA, 963 bp, Coffea arabica
atattatgccaaggttttaactgggagtcacataaatctggaagatggtatatggagcttcatcaaaaagctgctgagttatcatcacttggttttactgttgtctggttacctccgcccacagagtctgtttcacctgaaggctacatgccaaaggatttgtacaatctgaactccaggtaagaacagttgtgttggaaaactaatataggtactaagattagattagtttcctggaatcctgaatttggacatgcttatgtagttttcctgttcagataatttaccatatgtagaatgtaggactgaactctgactttagacctaaatgtgcaaacttcattaatactgtttgcacctacttcaatcctatgaactaacataatcatgaaaaccatggccttcttaatagtacaatctgctgtagtcttcttcatggatttgagttgaaaatgggtagctaaataaaatctattggggtagctgacacctgtctactttcagaagtaggctattcattcttcttttcatgcctgtctcccaattctttatgctactgggcagatgttaattaaattttttatattctcttgtaatttcttttttatctctaggtaaagttataatttgtttatttgattagaataactaaactaagggaacaaataaatctgattacttgaaattgctctgattattggaataaatgctgatcaacgaataactactgaaattgttataccacatcttcttgcagcactctccctctgtctgtctgcctctgtggttgatgtttttaacttcttgatcattttcagatatggaagtattgatgaattgaagtctctcgtgaagagattccatgaagttggcatcatggtcctcggagatgctgtgctaaaccatagatgtgcccattacaaaaatcagaatggtatttggaatatatttggaggtcgccta

SEQ ID NO: 31 - protein, 108 aa, Coffea arabica
ILCQGFNWESHKSGRWYMELHQKAAELSSLGFTVVWLPPPTESVSPEGYMPKDLYNLNSRYGSIDELKSLVKRFHEVGIMVLGDAVLNHRCAHYKNQNGIWNIFGGRL

SEQ ID NO: 32 - DNA, 810 bp, Gossypium hirsutum
atattgtgccaaggttttaactgggaatctaataaatctggacgatggtatatggaactcaaagaaaaagcttctgaaatatcttcacttgggtttactgtaatttggctgccaccaccaaccgagtctgtgtcacctgaaggttacatgccgaaggacttgtataatttaaattccaggtacatattgcaagcttgtagtttacataacaaatctttcaggtttttacattttggtgcctggtcaaattcaaagaagctgaaacttgcaaatatgaactttcaaatgctttttttgattcttttttctaaaaattataattgggaaactttgttcagggaaaaagatggaactgaatgttttttatgtttgctttagtatgctgatcaatggttggttgcaagtctcagattttggttcaaccaaaagaaagtgcacatttttttacctctcttttatgcttctttaacttacatttaaccatctgtaatagtaatataagacttcaattactcttttaatctaaaagactacatgcaaatgcctcttggacatgctgaaatgtagaaagtcatggttatttcccatatctgaaagaaatcatgttttcatgctcctcaagaggcctaagcaaaccagtgtgcaactgttttttcactgatcaggtatggaacaatcgatgagctgaaggacctcgtaaaaagcctccatggagttggtttgaaagttcttggtgatgttgtattgaatcaccgttgtgcacactatcaaaatcagaatggtgtttggaatctatttggtggacgccta

SEQ ID NO: 33 - protein, 108 aa, Gossypium hirsutum
ILCQGFNWESNKSGRWYMELKEKASEISSLGFTVIWLPPPTESVSPEGYMPKDLYNLNSRYGTIDELKDLVKSLHGVGLKVLGDVVLNHRCAHYQNQNGVWNLFGGRL

SEQ ID NO: 34 - DNA, 600 bp, Rosa woodsii
cctatgataaatttatactacaaaataatcttgcatgatcttcatagcttttactgaccccaaataaatcagctagctctacactgcatttttttgcagtaactttatacagaaaaaggccaacttttgttgggtaaaaaaaaaaaattaaggtggtttcgcaaacctctgcttgcgtggttatgcacttctaatcagttggaccaagcatgtactgaccatcaaagtttcagaccttatccttctttttccttttggtctgaactctgaagtcttgaagatgaacccttcttcttcttcactctctctcttttttttttggtttaccttggttggtagtaaattccactcctttgtttcgggagttccatttctttagtattcttaatattgttctgggagttccggagggatgctgtatacatcagtgattacttttgtttcacacttctcagatatggcactatggatgaactgaaggagactgtgaaggcattccataaagttggtatcaaagtacttggagatgttgttctaaatcaccgctgtgcgcagtatcagaatagcaatggtgtatggaatctatttggaggacgcctt

SEQ ID NO: 35 - protein, 48 aa, Rosa woodsii
YGTMDELKETVKAFHKVGIKVLGDVVLNHRCAQYQNSNGVWNLFGGRL

SEQ ID NO: 36 - DNA, 22 bp, Artificial Sequence
  Description of Artificial Sequence Made in lab
aacggtgattcagaacagcatc

SEQ ID NO: 37 - DNA, 22 bp, Artificial Sequence
  Description of Artificial Sequence Made in lab
atgtacccttgaggcgacacag

SEQ ID NO: 38 - DNA, 21 bp, Artificial Sequence
  Description of Artificial Sequence Made in lab
ggtgggaaccaaatcacagtg

SEQ ID NO: 39 - DNA, 35 bp, Artificial Sequence
  Description of Artificial Sequence Made in lab
gactcgagtcgacatcgattttttttttttttttt

SEQ ID NO: 40 - DNA, 17 bp, Artificial Sequence
  Description of Artificial Sequence Made in lab
gactcgagtcgacatcg

SEQ ID NO: 41 - DNA, 26 bp, Artificial Sequence
  Description of Artificial Sequence Made in lab
atattrtgccaaggttttaactggga

SEQ ID NO: 42 - protein, 24 aa, Artificial Sequence
  Description of Artificial Sequence Made in lab
WARRCGWCCWCCAAAWAKATTCCA

SEQ ID NO: 43 - DNA, 23 bp, Artificial Sequence
  Description of Artificial Sequence Made in lab
gttacttaccgggaaagctatac

SEQ ID NO: 44 - DNA, 18 bp, Artificial Sequence
  Description of Artificial Sequence Made in lab
gggcgctccatcaaaatc

SEQ ID NO: 45 - DNA, 21 bp, Artificial Sequence
  Description of Artificial Sequence Made in lab
tcttccacaggacctttattc

SEQ ID NO: 46 - DNA, 21 bp, Artificial Sequence
  Description of Artificial Sequence Made in lab
agtcctccggtacaagaagtc

SEQ ID NO: 47 - DNA, 26 bp, Artificial Sequence
  Description of Artificial Sequence Made in lab
ggaacaattgatgagctaaaagatac

SEQ ID NO: 48 - DNA, 19 bp, Artificial Sequence
  Description of Artificial Sequence Made in lab
ggaaatgagggtcatctgc

SEQ ID NO: 49 - DNA, 22 bp, Artificial Sequence
  Description of Artificial Sequence Made in lab
aacggtgattcagaacagcatc

SEQ ID NO: 50 - DNA, 22 bp, Artificial Sequence
  Description of Artificial Sequence Made in lab
atgtacccttgaggcgacacag

SEQ ID NO: 51 - DNA, 21 bp, Artificial Sequence
  Description of Artificial Sequence Made in lab
ggtgggaaccaaatcacagtg

SEQ ID NO: 52 - DNA, 2622 bp, Oryza sativa
atggcggtggcgagctggagcattccggcgatcccgcgggcgggtccgacagcgaggggtgtgctgctcggcggcgccttcgtgacggccgcgcggccgcccgtggcgtggcggtgccgggccacgctccctaggagagtgaggctcggtggcgtggtggcccgtgccggtgcggcggagacgccggtggccggctccggggaagccgggttgctgttctccgagaagttccccttgcgccgatcccgaacggtggaagggaaggcgtgggtgagggtcgatgcggagccggatggggagggcaagtgcaaggttgtgatcgggtgtgatgtagaggggaagtgggtgctgcattggggtgtctcctacgatggtgagcagggaagagaatgggaccaacctccttcagacatgagacctcccggttcagtgcctattaaggactatgcaattgaaacatctttggacactccacacaattcagaaggcaagacgattcatgaagtgcaaatcaaaattgataagggcacatcaattgctgctatcaattttgttctaaaggaagaggaaacaggtgcttggtttcagcacaagggtcaggatttcagaatacctttaagtggatcctttggtggagatctactaggaacagaacaagatattgatgtcaggccaggtcacctatctaacgtgttacagaaacctgagggacctattgctgagcctcataaaactgtacccgatgataaaggttcaagaaccaaacacatttcaggtttctatgaggaatacccaatcttaaaaacggtgtatgttcagaattttataactgttaatgtgagggaaaacaatggaacaactaaacatgctgtggaattcgacactgatattcctggagaagttatcattcattggggagtttgcaaagacaataccatgacatgggagatccccccagaaccacatccacctgcaacgaagatattccgacagaaagctcttcagaccatgctacaacaaaaagctgatggaacaggcaactctctatcattcttactggatggagagtattctggtctgatttttgtggtaaaacttgatgagtatacttggttgagaaatgtggagaatggatttgatttctacattcctcttacaagagcagacgccgaggctgacaaacagaaagccgatgataagtcttcacaagatgatggcttaatcagtgatataaggaatctggtggttgggctgtcgtctagaagaggtcagcgagcgaagaataaagttctgcaagaggatatcctacaagaaattgagaggttagcggcagaagcttatagcatttttaggagccccacaattgatactgtagaggaatctgtttacattgatgactcatccattgtgaagcctgcttgttctggtactggatctggatttgaaatattgtgtcaaggatttaactgggaatctcataagtcaggaaaatggtatgtggaacttggctcaaaggccaaggagttgtcatcgatgggtttcaccattgtctggtcaccaccacctactgattctgtgtcgcctgaaggatacatgccaagggatttgtataatctaaattccagatatgggaccatggaagagttgaaggaggctgtgaaacgttttcatgaagccggtatgaaggttcttggtgatgccgtcctgaatcacaggtgtgctcaatttcagaaccaaaatggcgtctggaatatttttggtggacgccttaactgggatgatcgagcagttgttgcagatgatccacatttccagggaagaggaaacaagagcagtggagataacttccatgcagccccaaacattgatcactcgcaagagtttgtgaggagtgatcttaaagaatggctttgttggatgagaaaggaagttggatacgatggatggcgacttgattttgttcgcggattttggggtggatatgtccacgattacttggaagcaagcgaaccatattttgcagtaggagagtactgggattctctcagttacacctatggtgaaatggattataatcaagatgcccacaggcagagaatagttgattggataaatgctacaaatggaactgctggtgcatttgatgttaccacgaaaggaatacttcactctgcactggaaagatctgagtactggcgtctgtctgatgaaaaaggaaaaccccctggagtgttaggttggtggccttcgcgtgctgtcacatttatagaaaatcatgacactggttctactcagggtcattggagattcccctttggtatggagttgcaaggctatgtctacatcttaactcacccaggcactcctgcaatcttctatgatcatatattttcgcatttacagccagagattgctaaattaatttctattagaaatcgccaaaagatccattgccgtagcaagatcaagatactgaaagcagagggaaatttatatgcggcagagattgatgagagggtaacaatgaagattggcgcaggacattttgagccaagcggccccacaaactgggtagttgctgccgagggacaggattacaaggtctgggaagtgtcatcgtag

SEQ ID NO: 53 - protein, 873 aa, Oryza sativa
MAVASWSIPAIPRAGPTARGVLLGGAFVTAARPPVAWRCRATLPRRVRLGGVVARAGAAETPVAGSGEAGLLFSEKFPLRRSRTVEGKAWVRVDAEPDGEGKCKVVIGCDVEGKWVLHWGVSYDGEQGREWDQPPSDMRPPGSVPIKDYAIETSLDTPHNSEGKTIHEVQIKIDKGTSIAAINFVLKEEETGAWFQHKGQDFRIPLSGSFGGDLLGTEQDIDVRPGHLSNVLQKPEGPIAEPHKTVPDDKGSRTKHISGFYEEYPILKTVYVQNFITVNVRENNGTTKHAVEFDTDIPGEVIIHWGVCKDNTMTWEIPPEPHPPATKIFRQKALQTMLQQKADGTGNSLSFLLDGEYSGLIFVVKLDEYTWLRNVENGFDFYIPLTRADAEADKQKADDKSSQDDGLISDIRNLVVGLSSRRGQRAKNKVLQEDILQEIERLAAEAYSIFRSPTIDTVEESVYIDDSSIVKPACSGTGSGFEILCQGFNWESHKSGKWYVELGSKAKELSSMGFTIVWSPPPTDSVSPEGYMPRDLYNLNSRYGTMEELKEAVKRFHEAGMKVLGDAVLNHRCAQFQNQNGVWNIFGGRLNWDDRAVVADDPHFQGRGNKSSGDNFHAAPNIDHSQEFVRSDLKEWLCWMRKEVGYDGWRLDFVRGFWGGYVHDYLEASEPYFAVGEYWDSLSYTYGEMDYNQDAHRQRIVDWINATNGTAGAFDVTTKGILHSALERSEYWRLSDEKGKPPGVLGWWPSRAVTFIENHDTGSTQGHWRFPFGMELQGYVYILTHPGTPAIFYDHIFSHLQPEIAKLISIRNRQKIHCRSKIKILKAEGNLYAAEIDERVTMKIGAGHFEPSGPTNWVVAAEGQDYKVWEVSS

SEQ ID NO: 54 - DNA, 8602 bp, Oryza sativa
atggcggtggcgagctggagcattccggcgatcccgcgggcgggtccgacagcgaggggtgtgctgctcggcggcgccttcgtgacggccgcgcggccgcccgtggcgtggcggtgccgggccacgctccctaggagagtgaggctcggtggcgtggtggcccgtgccggtgcggcggagacgccggtggccggctccggggaagccgggttgctgttctccgagaagttccccttgcgccgatcccgaacggtaacttgttctgctctgttgttgtgatctgcggcgaagctatgcttcgttgttttttttttgttggtgatggggtctgtggtgttcaggtggaagggaaggcgtgggtgagggtcgatgcggagccggatggggagggcaagtgcaaggttgtgatcgggtgtgatgtagaggggaagtgggtgctgcattggggtgtctcctacgatggtgagcagggaaggtactcgctgattctctctccgagctgtgtctctgtttgcatgatttatgaactatgagtgcatccttgttttgatcatccgtggggagttgatttgtgcattgttgagagcgtcgtttgaatcaacgatggttgtcagactggcaggtatgtcctcatggtaggctgctttccatatctatgtagcctcaccggtccagtctaagcacttgtatatgtatatagtgaaattactgtcaagtgtcaatgcaaccccttaatctttatttggcttcgattagaggaaattataaacaggttcattatgaaaaattcttacaaggtgggtgccattgatttatggctatacttggaaaatgcgcgcgaaaagagaattttgattaggtgacggggtagtgccgtaaccatgtaggttggctcttatcctgtttttcttttttatttggaatttcgatatagcgttgaagcgcgaggtgattagaaataggcatatctgcaagataggaatgataagttcttaaatttcaattgcaggttgcccggtgttatgtctccttatctcctttggagttgtcttgatattccaatattctatacgttacttaaatggcacgatagttttgcaaggaatatttagaattaatatttgtcatatcttttgtttttgtatgtattcaagcttcaacatacatacatttctccccgcaaaaaaaaaagaacatttctgtaaaaggcaaaaaattgaattttgtcgtctttcagatttagttgttgctgtaatgtccttccttgaatgcaaaaataaatgatctagctcctgaaatatatggcattctttatccttatttagtgacccaatttattgttatcacttgtttctataattttttgcagagaatgggaccaacctccttcagacatgagacctcccggttcagtgcctattaaggttgttctaatcctatgaaaaacatgttaaagaactcactactgacattcacatcccagggatctctaagtttgtttgatgtctgagtcgaaagctcgttgcatcctgtagccatattttttctcatttgatcttcctattgctgaacaatatttcaaactttcaggactatgcaattgaaacatctttggacactccacacaattcagaaggcaagacgattcatgaagtgcaaatcaaaattgataagggcacatcaattgctgctatcaattttgttctaaaggttcaaattttacgctgctgtatgctttctctgctgtcagttttatttcctaggcattctgtaccatgtgtcaaaaggcctagaagtctacgactggcccattagattcgtaaaattgctgaaagtgcctaaggtgggaagttgtcttcccattttttagttgtctaagaaaagaataatatataacccctacaggtatatctgcatactaaagtattgggagcggatatactaaacacatgcctcttagatctatagattagttgttaaatgcactactctttcttttaattactacatttttgcattcatcttttgatttgaatgtctcgatggactgctgctttgctaggaagaggaaacaggtgcttggtttcagcacaagggtcaggatttcagaatacctttaagtggatcctttggtggagatctactaggaacagaacaagatattgatgtcaggccaggttaggaagtattttagtgaatcatgttttagattattactatagtggtttggcactttgttcttttcattggttggtttttatgttatataaaaatgcatttccttctataagaaaataatcttatttagggttaaaatcttagtaattttcataggatgcttgatcatgaatattaccacattgaaaatttaccccatttgcacctttcatatttcagttatctaacatactcacatctctgatttcttaggggctttaggtcacctatctaacgtgttacagaaacctgagggacctattgctgagcctcataaaactgtacccgatgataaaggttcaagaaccaaacacatttcaggtttctatgaggaatacccaatcttaaaaacggtgtatgttcagaattttataactgttaatgtgagggaaaacaatggaacaactaaacatgctgtggaattcgacactgatattcctggagaagttatcattcattggggagtttgcaaagacaataccatgacatgggagatccccccagaaccacatccacctgcaacgaagatattccgacagaaagctcttcagaccatgctacaagtaaggaccttgatcccttggtagaggcaaagaaatgacatttaattgtatcaagctaaagtacaagataaatgtacttacaagctgtgctatataaaaattaacaagtaaaggtgttctggaaaagtaagtcaggctcatttgtatttttggtatgtcctgattctatctgcgctccaaaaacagggaattgtatcatttgcacttcttaggttgtctgaacttgaatttaccactatggcatatgcatatgtggtaaatcatgaaagaatgctcgctatagaaaaataaaaaaaggaacattttttaagtgtacagaaatgatttcacttttttcttattgcttaagaaaagtttaaaactcttctttgttttcctttttgaatggtaccttcaaatgatccattattaagagggttacttttagtctccctttgatcaggggaaatgtgcaaaactttatatcaagattcaagtcaacattttgaaaatctgaaggaaattgtgaagttctttgtttgtctccctactccattaccgacagactggcttctttttgggtttgatttcttcctttactagtctagtgagggcaagcattttcttatcagcaattctctttcaacaatcctacatcttcaatgtaggtgctgttttttggggatattttggtagtagtacactatgttctctaaaatcactgaaaatgcctcaattgatgccttttcatttttcaagatggttttgtattaatgttttcaatagtttcagctgtgggaaatcagttaagtatcactcttcacacatctcttatcatactgcagcaaaaagctgatggaacaggcaactctctatcattcttactggatggagagtattctggtctgatttttgtggtaaaacttgatgagtatacttggttgagaaatgtggagaatggatttgatttctacattcctcttacaagagcagacgccgaggctgacaaacagaaagccgatgataagtcttcacaagatgatggcttaatcagtgatataaggaatctggtggttgggctgtcgtctagaagaggtcagcgagcgaagaataaagttctgcaagaggatatcctacaagaaattgagaggttagcggcagaagcttatagcatttttaggagccccacaattgatactgtagaggaatctgtttacattgatgactcatccattgtgaagcctgcttgttctggtactggatctggatttgaaatattgtgtcaaggatttaactgggaatctcataagtcaggaaaatggtatgtggaacttggctcaaaggccaaggagttgtcatcgatgggtttcaccattgtctggtcaccaccacctactgattctgtgtcgcctgaaggatacatgccaagggatttgtataatctaaattccaggtatatctctgtgtgcacaattattgtgttactttagtaatgttttctatgaaatttgaaatattagtaatattggtgctaagctggaagtttacacttccgctatctacaaaaaaatgagatgggatagttggtaagttagagataaagctagtaaccaagttagagctaagatggtgctatgctccagcttgttttatgtaaagccatgctgtgtacatgagttgattatgatggataatcagtctaccctaccttaagtttccttcttctccgttgccaaagcttcttcctccttgctgtttccaaaccaatctactgctaattctccattgcctacctcagctaaccttctacaatctagcatatagcacttgtgctgaagattttagtactacgctatctagtagaaacgaaaacctgggtagcatatgaaattcacatatggattctaaaggagaaaaccccttttatacccctaaaatctctattaaaggcactattactgctgcagtagcttgttgttcttctatggttaacactaccaccactatgcaataaatatttatatgtcgcttgaggcatcacttgtccactatgatgcatggatacgcgatactggtacggcgatacgagattttttaaaaaacatcaatacgtcgatacggcgagtatatataaaaatatgtttaaaaataaaatatagggaactacaaaagggtaaagcaataagattatcttaaaatatattctatgaattgttgcctgttacaaaaaatatagcaaatatagccttgtattacaaatatagcaagtatattaaagttataaatatatcagaagtatagcattgggtctttaatgtgctattgaacaaggaggttacactgctaggtatcaaagaagtatctgataagtatcgtgtgagtatcggtatttattttttcttttttttccattgtttaatgaccgatactcctccgataccgtatcgacagagtatcggacgtatcgccgtatcggataccggtacggcaagaaattgaagtatcggtgcatcatagcttgtccagaaaactggctctcatcctacttgattttgactacccttatgaagcttcttagttgttgtcttccttgtttccaaaaaaaaaaaaaaaacaatgattttcaaagggaaaattatgacactgaaagatctatgcctttatacagcataatagtatgaacccatcatgtcagctcatgccattcattaatatgatatcatcaatgtttcagatatgggaccatggaagagttgaaggaggctgtgaaacgttttcatgaagccggtatgaaggttcttggtgatgccgtcctgaatcacaggtgtgctcaatttcagaaccaaaatggcgtctggaatatttttggtggacgccttaactgggatgatcgagcagttgttgcagatgatccacatttccaggtgaggttccaccttcccttgcaaactatttaattagaattatttgaaaatgtcattcatgaatgttcattatgttaactaagtatcacaacattatgattttcttcatgaactgctggtttcatttgcacaaatattttcctccagtggagcttgataaagttatgcgaaaataagcttaatttaaccaacaaatgatgcttggagatagtctcattgagtgacctacaaactttgatagactccaccttttcaacttgcatacatttagtattggaagtaaacacagattagcatccgaaataacatagaaattgacatagttccaaaatggaaaaaagacatactcaaacttcagcagccatgaacattgatcaatcataatattgttctacatgcctattccatttcattttacttaacaatgcactatgtgatgtactagacttactttctaatttttgggtaaaactatatgtccttgtagggaagaggaaacaagagcagtggagataacttccatgcagccccaaacattgatcactcgcaagagtttgtgaggagtgatcttaaagaatggctttgttggatgaggtatgctatcttgttttttcttggctaaaactccttttgttgctcatgcttaactatatatgtgtattccccatgcgaagggttgtatgttatagcatccccatgcaacagattaaatagatttgatgtacatacattgtttctggatggcgtgtttagctgagtgctagttttctttcctcatatcacattcttttcaaagctatgttttcatatatgttagtttaccttgctcttaccacaaaatggaatgtacacagtgcacatcctcatcaatatcacaagattctttttcatgctacgataataggtatatattgtaggctgctaagctcctataataacaaactggattggacaaatgctacacctgaatatctgcaaattgtcatgggttccaccatatatatatgtatatatatttttgccatgtagcttagggatgatcgattctgtatctatgtaattgtgtagtgcttggacatatgcaaaaaaaaggggaagatattgcaggagctttaccctcattaatactgaaactatttcttggagccaatttattagggagtattgttgggaacagtagcttactagtcctaatgtggttgctgcatattttcattagctgaacaggataaattaaatcatagaacatagcattagagcagcattcctgaaaacgttagcatcagatgacttcattgtatgtttgcttctgcttgacttgcagaaaggaagttggatacgatggatggcgacttgattttgttcgcggattttggggtggatatgtccacgattacttggaagcaagcgaaccatattttgcagtaggagagtactgggattctctcagttacacctatggtgaaatggattataatcaagatgcccacaggcagagaatagttgattggataaatgctacaaatggaactgctggtgcatttgatgttaccacgaaaggaatacttcactctgtgagactccattttcttaccaagttttataatagtgcttatgggtgatttagtatttttgaaactctagaatttatatgcctttccatggtttgagcttctcagttcggatatgcatcatgaagaatttcctgccctttagcatttttcttttgggtggaggtatactttgtccttcaattatttgataattaaagttttattgctgctatgaagacgcagcatgctgaaccacattaattaaacttgcatctttggcagtattcctgttccatggcaatatttaaagtctcattaatctgttgtaaagtggttttctgatgatagttaactcatccatgattgtaatagccaattctgccagtttgagaaggatgctattgctctggattagccatgcatattttgtcttacacatcttttctggtatacaggcactggaaagatctgagtactggcgtctgtctgatgaaaaaggaaaaccccctggagtgttaggttggtggccttcgcgtgctgtcacatttatagaaaatcatgacactggttctactcaggtacattacaaaatccctacatacacaaataagtattactccgtagcattatcttcagaaggttactatatggcagcatgattctggcaaatctgacatcatatttttgttatatttgcatagaaacatcacaaatgtgcaacgacctgcatgcatttcttaataatagtcagatagttgtccctttttttgaacgctgcttaatttcttgttttcaaacaactaaattttgctcagcccatccttgtagaactcttttgaaactggaatgcttttatggatactaatcaaattataactggagctgttaagctgtgttcgaacaaaatcatggctaactttctttgatatctttgtagggtcattggagattcccctttggtatggagttgcaaggctatgtctacatcttaactcacccaggcactcctgcaatcttctatgatcatatattttcgcatttacagccagagattgctaaattaatttctattagaaatcgccaaaagatccattgccgtagcaaggtaaaacttcctcttgtatcgtatggtaccccctcttaccctccttgcccttccccactctattcatgttctctaatgcttgaatttatataacatttacagatcaagatactgaaagcagagggaaatttatatgcggcagagattgatgagagggtaacaatgaagattggcgcaggacattttgagccaagcggccccacaaactgggtagttgctgccgagggacaggattacaaggtctgggaagtgtcatcgtag

SEQ ID NO: 55 - DNA, 4486 bp, Arabidopsis thaliana
atgtccactgttcccattgagtctcttctccaccattcttatcttcgtcacaactcaaaagtcaatcgcggaaatagaagcttcatacctatctcgttgaatctccgttctcatttcacttctaacaaactactacactcaattggaaaaagtgtcggtgttagctcgatgaacaaaagtcccgtcgccattcgtgctacctcatcggatactgccgtcgtggaaaccgctcaatcggatgatgtcatcttcaaggaaattttccctgttcagcgaatcgaaaaggttttctcaatcgccgtacacagtcacatttagcggaaattgattgaatatagttcctatgttgttgttagttaaatctaataaacacattgattttgtgtatgtgttttgctgaatttaatggttttgatttcgatttttgggaatgataggcagaaggaaagatttatgttcgattaaaggaagtgaaggagaagaattgggagctgagtgttggatgtagtatacctggaaaatggattttgcattggggagtttcatatgtgggtgatactggcaggtcctttcctcatttacctcttttttttggttgtctcatttacattattgagaaaattttattcccttgatggtctctacattttcgattcgatagtgaatgggatcaacctccggaagatatgagacctcctggctcaattgccattaaggtattactctggtgtcttttttcccttttggtttatatctactttggatagttgtaatatgttgttatgtggtgtatcaggactatgccatagagacacctttgaagaagttatctgaaggagattctttttttgaagttgctattaatctaaatctggagagttctgtagcagctctcaattttgttttgaaggttctgctgcttttaacctttgtaacaaagttgattgttggtttggctttagttgtgcatttatttgggactgtttgtgttccaggacgaagaaactggggcgtggtatcagcacaaagggagagactttaaggttcctcttgtagacgatgttcctgataatggaaatttgatcggagctaagaaaggatttggtaaatggccttttttttatatatatatatttgtcaatagcttctactattgtccggactacagattttgcattcctatagttaggcattctttcacctcctcattcttttttaacgatgtataatgacggaagttagtattgcaacagactttctttgaatctttaaactgattaaggcttgctttctcttggatctgtaggtgcccttgggcagctttcaaacatccctctcaaacaagataagtccagtgcagagactgattctattgaagaaagaaaaggtcttcaagagttttacgaggagatgccaataagtaaacgtgttgccgatgataactcagtcagtgtcactgcaaggaaatgccctgaaacatctaagaacattgtatcaattgaaaccgatttaccgggtgacgttactgtccactggggagtttgcaaaaacggcactaagaaatgggaaattccatctgaaccttaccctgaagagacatctttgtttaagaacaaggcattacgcactcgtttacaggtacatgtagaagtaggcaagagtcacatgttcagatttgaaaatttggcaagaatcacttgtgttgtgtctctgtaattttagtctgtgagattcaaaatcagtttttggttagcacctggcgtattcgcaattgtccaagcaatttgtaggttgtgcaggcctggtttttaattaagcattagaatggttagcaacccaatgcattcggttcttctgttttttgcgcaccctgatagacatttccttcatacttttaccacatgatgccgtcactatgtctttgctgctgcttccatatatcttttgggcacctagagatcattttcatgttggcacctaaatattttgtctagcttacctattagaatattttctgtgtctatcagtgacattacctaagtgtttatgcatatgaacattctgcagcgaaaagacgatggaaatggatcgtttggattattctctctggatggaaagcttgaagggttatgctttgttcttaagttaaatgaaaatacttggctaaattataggggggaagacttctatgtccctttccttacttcaagtagctcgcccgttgaaactgaagctgcccaagtgagtaaacccaaacgaaaaacagataaagaagtgtctgctagtggatttactaaagaaatcatcacggagataaggaacttggcaattgacatttcctctcataagaatcagaagacaaacgtcaaagaagtgcaggaaaacattctacaagaaattgagaaactggctgcggaggcatatagcatatttagaagcacaactccagctttttccgaggaaggtgttttagaagcagaagctgacaagcctgacattaaaatctcctcaggaaccggctcgggatttgagatattatgccaaggtttcaactgggagtccaataaatctgggagatggtacttggaacttcaagaaaaagccgatgagttagcttcacttggattcactgttctgtggttacctccaccgacagaatctgtgtcacctgaagggtacatgcctaaagacctgtataacttgaattccaggtgagttccgcatttttatcagactgtcttttgtagcttagttgcttaaacttcgttatggattgttgaatcataaagtagaattctggttaatgttgtttttgttgccttaaatgcatgatcagccagcagtggaaatgttaattctcttaggagattaacaatacttatgcatttacatgatatcatgtatctgaagtacatgtttccccctattttttgttgcaaactttcagatatggaacaattgatgagctaaaagatacagtgaagaaatttcacaaggtcgggatcaaagttttaggcgatgctgttctgaatcaccgctgtgcacacttcaaaaatcaaaatggtgtatggaatctatttggaggacgtctaaactgggacgatagggcagtagttgcagatgaccctcatttccaggtattactttcctttctttggatttaaaataaacagctatagaacgaaaacgaggcaaattttttgatagagaatgtgctttttgctgttatctgctgccgtattatgaacttggactagtgtttttgagagaaataaggtgcatatgatctctattacccttgatgatacaatcaactaattcattgattcagggtagaggaaacaagagcagtggagataatttccatgctgctccaaacatagatcactcgcaagactttgttaggaaggatatcaaggaatggctatgctggatgatgttagttcccctgttatttgtttaatatcttcttctgattagtttggtgtctgatttctttctctattttgatttcacctgcagggaagaagttgggtatgatggatggaggcttgactttgtaagagggttctggggaggttatgtgaaagactatatggatgctagcaaaccgtactttgcggttggtgaatattgggattcgttaagttacacgtacggagaaatggactacaatcaagacgcacatcgtcaaagaatagttgactggattaatgcaactagtggagctgctggtgcatttgatgtcactaccaaaggaattcttcatacggtactaataatatcgtatcttgttacgtttgatacaacatttccttttggtatataatatctggtagctttctgtgtgtaggcgcttcaaaaatgtgaatattggagactctcagacccaaaagggaagcctccaggtgtagttggttggtggccttctcgtgctgtaacattcatcgagaatcatgacactggctctacacaggttttatttctcagcattctaagtacctcaagtttctgttctctataaataaaccgtcttaacaatttcttccgcttttcttaaagggtcattggagatttccggaggggaaggaaatgcaaggatatgcttacatcctaactcatccaggaacaccagcggtcttcttcgaccatatcttctcggattatcattccgagattgctgcacttctctctctcaggaacagacagaaactccactgtcggagtgaggtaaagtagtaatcaagaacttacaatctttatacaaaatttcgacgggtcttaaaaccagcctcctttgtcttccttgcttttgtgaattataaaggtgaatatagacaagagtgagagagatgtgtatgcggctataatagatgaaaaggttgcaatgaagatcggaccagggcattatgaaccaccaaacggatcgcaaaactggtctgtagccgttgaaggcagagactacaaggtgtgggaaacatcttaa